(19) United States
(12) Patent Application Publication
(10) Pub. No.: US 2022/0315991 A1
(43) Pub. Date: Oct. 6, 2022

(54) Detecting Salmonella from an Environmental Sample

(71) Applicant: Neogen Corporation, Lansing, MI (US)

(72) Inventors: Preetha Biswas, Okemos, MI (US); Robert S. Donofrio, Williamston, MI (US); Lei Zhang, Okemos, MI (US); Lin L. Walker, Raleigh, NC (US); Andrew Michael Laseck, East Lansing, MI (US); Debra Lynn Foti, Fenton, MI (US); Wesley David Colangelo, Howell, MI (US); Vimukthi A.A. Senevirathne, Lansing, MI (US); Ariel Fox, East Lansing, MI (US); Jolien Weytens, New Lathrop, MI (US)

(21) Appl. No.: 17/218,294

(22) Filed: Mar. 31, 2021

(51) Int. Cl. C12Q 1/689; G01N 1/34; G01N 21/64

(52) U.S. Cl. CPC C12Q 1/689; G01N 1/34; G01N 21/6428; G01N 2021/6439

(57) ABSTRACT

The present disclosure provides compositions, methods and kits for detection of Salmonella from an environmental sample, without the need for an enrichment or prolonged incubation step.

### REFERENCE TO THE SEQUENCE LISTING

The instant application contains a Sequence Listing which has been submitted electronically in ASCII format and is hereby incorporated by reference in its entirety. Said ASCII copy, created on Mar. 28, 2021, is named 234994_461526_SL.txt and is 2,133 bytes in size.

### TECHNICAL FIELD

The present disclosure relates to the detection of Salmonella from an environmental sample.

### BACKGROUND

Bacterial contamination and infection can pose a serious problem for public health. For example, Salmonella is a harmful pathogen that can be especially problematic in the food industry. There is a need for methods and tools to rapidly detect Salmonella in environmental samples, including for food safety testing and environmental monitoring.

### SUMMARY OF THE INVENTION

The present disclosure provides, in part, compositions, methods, and kits for rapidly detecting Salmonella in samples, such as environmental samples. In various aspects, the provided compositions, methods, and kits enable determining presence or absence of Salmonella in environmental samples without enrichment or prolonged incubation prior to performing the detection assay itself. Among other things, a benefit of the provided methods is that results can be obtained in a short time (e.g., about 1-2 hours from sample collection), compared to conventional methods that may take 24 hours or more. Due to low sensitivity, conventional methods require enrichment of the sample to increase the concentration of target organisms, or other types of prolonged incubation steps required to increase the concentration of target molecules to detectable levels. The present disclosure provides improvements and benefits compared to such conventional methods.

In a first aspect, the present disclosure provides a method for determining the presence or absence of target bacteria in a sample, comprising the steps of: (i) providing a sample; (ii) contacting an aliquot of the sample with a lysis mixture under conditions to lyse at least a portion of cells in the aliquot, thereby generating a lysate; (iii) contacting an aliquot of the lysate with a detection mixture, thereby generating an assay mixture; (iv) in the assay mixture, reverse transcribing a target RNA of the target bacteria to form target cDNA and amplifying the target cDNA by helicase-dependent amplification (HDA); and (v) detecting presence or absence of the amplified target cDNA, thereby determining the presence or absence of the target bacteria in the sample. The aliquot of the sample may be a portion of the sample. The aliquot of the lysate may be a portion of the lysate. The HDA may be thermostable helicase-dependent amplification (tHDA).

In some embodiments, the method does not include enrichment for cells in the sample. In some embodiments, the method does not include a prolonged incubation period to increase concentration of the target RNA prior to contacting the sample with the lysis mixture.

In some embodiments, the sample comprises bacteria that is not the target bacteria. In some embodiments, the sample is an environmental sample. In some embodiments, the sample is collected from an environment comprising a low concentration of the target bacteria cells. In some embodiments, the method is of sufficient sensitivity to detect the presence of the target bacteria from a sample having as little as about 30-60 colony forming units (CFU) of the target bacteria.

In some embodiments, the sample is taken from a surface that is a solid, and the concentration of the target bacteria cells on the surface is a low concentration. In some embodiments, the solid surface further comprises microflora that is not the target bacteria. In some embodiments, (i) the solid surface comprises from about 10 to about 200 CFU of the target bacteria per 1 square inch of the solid surface; (ii) the solid surface comprises from about 10 to about 100 CFU of the target bacteria per 1 square inch of the solid surface; or (iii) the solid surface comprises from about 10 to about 50 CFU of the target bacteria per 1 square inch of the solid surface.

In some embodiments, the target bacteria is of the genus Salmonella and the target RNA comprises a Salmonella RNA sequence. In some embodiments, the target RNA comprises a Salmonella RNA sequence of the 23 S ribosomal RNA.

In some embodiments, the method further comprises collecting the sample to be provided in step (i), wherein the sample is collected from an environment that is being tested for bacterial contamination by the target bacteria. In some embodiments, the sample is collected with a pre-moistened swab or sponge.

In some embodiments, the method further comprises a step of pre-treating an aliquot of the sample to remove nucleic acids not associated with intact cells, thereby generating a pre-treated sample; and the step of contacting the aliquot of the sample with the lysis mixture comprises contacting an aliquot of the pre-treated sample with the lysis mixture under conditions to lyse at least a portion of cells in the aliquot of the pre-treated sample. The aliquot of the pre-treated sample may be a portion of the pre-treated sample. In some embodiments, the step of pre-treating the aliquot of the sample comprises contacting the aliquot of the sample with a pre-treatment mixture under conditions to remove nucleic acids not associated with intact cells in the aliquot of the sample. In some embodiments, the pre-treatment mixture comprises a nuclease that cleaves nucleic acids and the lysis mixture comprises a component that inactivates the nuclease. In some embodiments, the nuclease is micrococcal nuclease. In some embodiments, the pre-treatment mixture further comprises a divalent salt and the component that inactivates the nuclease is a divalent ion chelator. In some embodiments, the pre-treatment mixture is lyophilized. In some embodiments, the lysis mixture comprises at least one lytic enzyme and at least one protease. In some embodiments, the lysis mixture further comprises Chelex-100.

In some embodiments, the step of detecting presence or absence of the amplified target cDNA comprises: (i) detecting a level of the amplified target cDNA above a threshold level and thereby determining the presence of the target bacteria in the environmental sample; or (ii) detecting a level of the amplified target cDNA below a threshold level and thereby determining the absence of the target bacteria in the environmental sample. In some embodiments, the step of detecting comprises measuring a fluorescent readout indicative of the presence of the amplified target cDNA.

In some embodiments, the detection mixture comprises at least one probe for detecting the amplified target cDNA. The probe may be a fluorescent molecular probe. In some embodiments, the probe is a conditional fluorescent hybridization probe that emits fluorescence when hybridized to a nucleic acid molecule comprising the nucleic acid sequence of: (i) CAC GTA GGT GAA GTG ATT TAC TCA CGG (SEQ ID NO: 6), or a sequence with at least 90% sequence identity to SEQ ID NO: 6; or (ii) CAC GTA GGT GAA GIG ATT TAC TCA TGG (SEQ ID NO: 7), or a sequence with at least 90% sequence identity to SEQ ID NO: 7. In some embodiments, the probe comprises the nucleic acid sequence of: CCA TGA GTA AAT rCAC TTC ACC TAC GTG (SEQ ID NO: 5), or a sequence with at least 90% sequence identity to SEQ ID NO: 5. In some embodiments, the detection mixture further comprises RNase H2. In some embodiments, the detection mixture further comprises a helicase, an energy source for the helicase, a DNA polymerase, a reverse transcriptase, and dNTPs. In some embodiments, the detection mixture further comprises a single-stranded binding protein. In some embodiments, the detection mixture further comprises a control RNA and a control probe that is able to detect amplification products of the control RNA. In some embodiments, the detection mixture further comprises a first primer having hybridization specificity for a single-stranded nucleic acid region comprising a nucleic acid sequence of the target RNA and a second primer having hybridization specificity for a single-stranded nucleic acid region comprising a nucleic acid sequence complementary to the target RNA sequence. The first primer may comprise the nucleic acid sequence of 5′ CTG ACT TCA GCT CCG TGA GTA AAT 3′ (SEQ ID NO: 3) or a sequence with at least 90% sequence identity to SEQ ID NO: 3 and the second primer may comprise the nucleic acid sequence of 5′ GAG AAG GCA CGC TGA CAC 3′ (SEQ ID NO: 2) or a sequence with at least 90% sequence identity to SEQ ID NO: 2. In some embodiments, the detection mixture further comprises (i) an enzyme that binds uracil in a DNA strand and converts it into an apurinic site; (ii) an enzyme that cleaves DNA at apurinic sites; and (iii) a specialized dNTP that is recognized by the enzyme of (i). In some embodiments, the enzyme that binds uracil in a DNA strand and converts it into an apurinic site is a uracil DNA glycosylase, the enzyme that cleaves DNA at apurinic sites is Endonuclease VIII and the specialized dNTP that is recognized by the enzyme of (i) is dUTP.

In some embodiments, at least one of the lysis mixture and the detection mixture is lyophilized.

In some embodiments, the time from providing the environmental sample to detecting at least some of the amplified target cDNA is 2 hours or less. In some embodiments, the provided environmental sample has been stored at 4° C. for up to 24 hours.

In a second aspect, the present disclosure provides a kit comprising a first mixture and a second mixture, wherein the first mixture comprises micrococcal nuclease and a divalent salt and the second mixture comprises a divalent ion chelator, at least one lytic enzyme and at least one protease. In some embodiments, the divalent salt is calcium chloride (CaCl₂) and the divalent ion chelator is EGTA. In some embodiments, the at least one lytic enzyme comprises lyosozyme and mutanolysin and the at least one protease is proteinase K.

In some embodiments, the kit further comprises a third mixture, said third mixture comprising a chelating resin. The chelating resin may be Chelex-100.

In some embodiments, the kit further comprises a fourth mixture, said fourth mixture comprising a helicase, an energy source for the helicase, a DNA polymerase, a reverse transcriptase, and dNTPs. In some embodiments, the fourth mixture further comprises a single-stranded binding protein. In some embodiments, the fourth mixture further comprises (i) an enzyme that binds uracil in a DNA strand and converts it into an apurinic site; (ii) an enzyme that cleaves DNA at apurinic sites; and (iii) a specialized dNTP that is recognized by the enzyme of (i). In some embodiments, the fourth mixture further comprises a first primer having hybridization specificity for a single-stranded nucleic acid region comprising a nucleic acid sequence of the Salmonella 23S ribosomal RNA and a second primer having hybridization specificity for a single-stranded nucleic acid region comprising a nucleic acid sequence complementary to the nucleic acid sequence of the Salmonella 23 S ribosomal RNA. The first primer may comprise the nucleic acid sequence of 5′ CTG ACT TCA GCT CCG TGA GTA AAT 3′ (SEQ ID NO: 3) or a sequence with at least 90% sequence identity to SEQ ID NO: 3 and the second primer may comprise the nucleic acid sequence of 5′ GAG AAG GCA CGC TGA CAC 3′ (SEQ ID NO: 2) or a sequence with at least 90% sequence identity to SEQ ID NO: 2. In some embodiments, the fourth mixture further comprises at least one probe. In some embodiments, one of the at least one probe is a conditional fluorescent hybridization probe that emits fluorescence when hybridized to a nucleic acid molecule comprising the nucleic acid sequence of: (i) CAC GTA GGT GAA GTG ATT TAC TCA CGG (SEQ ID NO: 6), or a sequence with at least 90% sequence identity to SEQ ID NO: 6; or (ii) CAC GTA GGT GAA GTG ATT TAC TCA TGG (SEQ ID NO: 7), or a sequence with at least 90% sequence identity to SEQ ID NO: 7. In some embodiments, one of the at least one probe comprises the nucleic acid sequence of: CCA TGA GTA AAT rCAC TTC ACC TAC GTG (SEQ ID NO: 5), or a sequence with at least 90% sequence identity to SEQ ID NO: 5.

In some embodiments, the first, second, third and/or fourth mixture is lyophilized. In some embodiments, the first mixture is lyophilized and upon resuspension of the first mixture, the concentration of micrococcal nuclease ranges from 0.1-0.3 Units/μL and the concentration of the divalent salt ranges from 2-6 mM. In some embodiments, the second mixture is lyophilized and upon resuspension of the second mixture, the concentration of the divalent ion chelator ranges from 2-5 mM.

In some embodiments, the kit is for use in a method of determining the presence or absence of target bacteria in an environmental sample.

In a third aspect, the present disclosure provides a kit comprising a first primer having hybridization specificity for a single-stranded nucleic acid region comprising a nucleic acid sequence of Salmonella 23S ribosomal RNA and a second primer having hybridization specificity for a single-stranded nucleic acid region comprising a nucleic acid sequence complementary to the nucleic acid sequence of the Salmonella 23S ribosomal RNA.

In some embodiments, the first primer comprises the nucleic acid sequence of 5′ CTG ACT TCA GCT CCG TGA GTA AAT 3′ (SEQ ID NO: 3) or a sequence with at least 90% sequence identity to SEQ ID NO: 3 and the second primer comprises the nucleic acid sequence of 5′ GAG AAG GCA CGC TGA CAC 3′ (SEQ ID NO: 2) or a sequence with at least 90% sequence identity to SEQ ID NO: 2.

In some embodiments, the kit further comprises at least one probe for detecting a nucleic acid molecule comprising the nucleic acid sequence of: CAC GTA GGT GAA GTG ATT TAC TCA CGG (SEQ ID NO: 6), or a sequence with at least 90% sequence identity to SEQ ID NO: 6.

In some embodiments, the kit further comprises at least one probe for detecting a nucleic acid molecule comprising the nucleic acid sequence of: CAC GTA GGT GAA GTG ATT TAC TCA TGG (SEQ ID NO: 7), or a sequence with at least 90% sequence identity to SEQ ID NO: 7.

In some embodiments, the kit further comprises at least one probe that comprises the nucleic acid sequence of: CCA TGA GTA AAT rCAC TTC ACC TAC GTG (SEQ ID NO: 5), or a sequence with at least 90% sequence identity to SEQ ID NO: 5.

In some embodiments, the first primer, the second primer and the probe are lyophilized.

In a fourth aspect, the present disclosure provides a primer comprising or consisting of the nucleic acid sequence of GAG AAG GCA CGC TGA CAC (SEQ ID NO: 2) or a sequence with at least 90% sequence identity to SEQ ID NO: 2.

In a fifth aspect, the present disclosure provides a primer comprising or consisting of the nucleic acid sequence of CTG ACT TCA GCT CCG TGA GTA AAT (SEQ ID NO: 3) or a sequence with at least 90% sequence identity to SEQ ID NO: 3.

In a sixth aspect, the present disclosure provides a composition comprising at least one of: (i) a primer comprising or consisting of the nucleic acid sequence of GAG AAG GCA CGC TGA CAC (SEQ ID NO: 2) or a sequence with at least 90% sequence identity to SEQ ID NO: 2; and (ii) a primer comprising or consisting of the nucleic acid sequence of CTG ACT TCA GCT CCG TGA GTA AAT (SEQ ID NO: 3) or a sequence with at least 90% sequence identity to SEQ ID NO: 3.

In a seventh aspect, the present disclosure provides a lyophilized composition comprising micrococcal nuclease and calcium chloride (CaCl₂), wherein, upon resuspension of the lyophilized composition, the concentration of micrococcal nuclease ranges from 0.1-0.3 Units/μL and the concentration of CaCl₂ ranges from 2-6 mM. In some embodiments, upon resuspension of the lyophilized composition, the concentration of micrococcal nuclease is 0.22 Units/μL and the concentration of CaCl₂ is 4.1 mM.

In an eighth aspect, the present disclosure provides a lyophilized composition comprising (i) at least one of lyosozyme and mutanolysin; (ii) at least one of proteinase K and achromopeptidase; and (iii) EGTA, wherein, upon resuspension of the lyophilized composition, the concentration of lysozyme ranges from 0-1 mg,/mL, the concentration of mutanolysin ranges from 0-30 Units/mL, the concentration of proteinase K ranges from 0-1 mg/mL, the concentration of achromopeptidase ranges from 0-150 Units/mL, and the concentration of EGTA ranges from 2-5 mM. In some embodiments, upon resuspension of the lyophilized composition, the concentration of lysozyme is 0.8 mg/mL, the concentration of mutanolysin is 20 Units/mL, the concentration of proteinase K is 0.8 mg/mL, the concentration of achromopeptidase is 85.6 Units/μL, and the concentration of EGTA is 2.6 mM.

### BRIEF DESCRIPTION OF THE DRAWINGS

These and other features, aspects, and advantages of the present disclosure may be better understood when the following detailed description is read with reference to the accompanying drawings.

FIG. 1 shows an illustrative sequence of steps for a Salmonella detection method of the present disclosure. The method comprises pre-treatment, lysis and a Salmonella detection assay. Details of each of these steps is further described elsewhere by the present disclosure, in some cases, as separate methods of pre-treatment, lysis and nucleic acid amplification and detection, respectively, which can be combined in the sequence shown. The illustrative method comprises pre-treating an aliquot of a sample (e.g., a provided environmental sample) to remove nucleic acids not associated with intact cells, thereby generating a pre-treated sample; contacting an aliquot of the pre-treated sample with a lysis mixture comprising lytic enzymes under conditions to lyse at least a portion of cells in the aliquot of the pre-treated sample, thereby generating a lysate; contacting an aliquot of the lysate with a detection mixture comprising components for amplification and detection of Salmonella target nucleic acids; and performing the Salmonella detection assay.

FIG. 2A-FIG. 2G are graphs showing the detection of Salmonella enterica subspecies and serovars with primers and probe designed for amplification and detection of Salmonella nucleic acids.

FIG. 3A-FIG. 3B are graphs showing amplification and detection results of exclusive bacterial strains with primers and probe designed for amplification and detection of Salmonella nucleic acids.

FIG. 4 is a graph showing amplification signals observed for S. typhimurium.

FIG. 5 is a graph showing amplification signals observed for no template control (NTC) samples that did not have Salmonella cells present.

### DETAILED DESCRIPTION

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference and understanding, and the inclusion of such definitions herein should not necessarily be construed to mean a substantial difference over what is generally understood in the art. Articles such as “a,” “an,” and “the” may mean one or more than one unless indicated to the contrary or otherwise evident from the context. The term “and/or” means one or all of the listed elements or a combination of any two or more of the listed elements. As appropriate, procedures involving the use of commercially available kits and/or reagents are generally carried out in accordance with manufacturer's guidance and/or protocols and/or parameters unless otherwise noted. The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

About: The word “about” is understood to modify numbers recited in the specification and claims whether or not explicitly stated. The term “about” is intended to take into account standard measurement errors and encompass rounding off.

Aliquot: An “aliquot” as used with reference to an aliquot of a sample or lysate means a portion or all of the stated sample or lysate that is used for further processing, such as downstream steps in a method.

Control RNA: As used herein, the term “control RNA” refers to an RNA molecule added to a detection mixture or an assay mixture for purposes of serving as a positive internal control for amplification and/or detection by methods described herein. A control RNA can be detected with a control probe. As used herein, a “control probe” is a probe that is designed to detect amplification products of the control RNA.

Downstream methods: As used herein, the phrase “downstream methods” or “downstream assays” refers to one or more additional methods (e.g., one or more additional steps) or assays (e.g., a detection assay) that are carried out. The one or more additional methods or assays may be carried out after the stated procedure is initiated, or after the stated procedure has been performed. For example, a method that is downstream to cell lysis may be carried out after lysis begins, and/or after a lysis step generates a lysate. A downstream method or downstream assay does not necessarily follow directly after the stated procedure. For example, additional intervening steps may be included between the stated procedure and the downstream method or assay.

Enrichment: The term “enrichment” refers to expansion of target cells (e.g., target bacteria cells). Enrichment may comprise exposing a sample comprising target bacteria cells to cell growth conditions that promote increasing the number of the target bacteria cells. Some methods of enrichment comprise use of a broth, which may be referred to as an “enrichment broth.” An enrichment broth may comprise both beneficial compounds for the growth of the target microbe (e.g., target bacteria), as well as inhibitory compounds for other microbes that are detrimental to the growth of the target microbe or to downstream assay steps. Continuing enrichment of the target microbe on different selective media is a known method of assaying for the presence of the target microbe. Methods known in the art, for example for food samples or food production environments or pharmaceutical environments, can be found in the USDA Microbiology Laboratory Guidebook (USDA-MLG), or the U.S. Food and Drug Administration (FDA) Bacteriological Analytical Manual (FDA-BAM).

Environmental sample: As used herein, the term “environmental sample” refers to a sample collected from the environment. In preferred embodiments, an environmental sample is collected from the environment in a room or a building where foods and/or beverages are produced and/or processed, such as that of a manufacturing plant or a commercial kitchen. In some preferred embodiments, environmental samples may be collected from food/beverage production, processing and/or service sites. Environmental samples may be collected from both food contact surfaces (e.g., slicers, mixers, utensils or conveyors) and non-food contact surfaces (e.g., floors, drains, carts or equipment housing). Environmental surfaces may be composed of a variety of materials or combinations of materials, such as stainless steel, plastic, ceramic tile, sealed concrete, or rubber. In some embodiments, environmental samples may be collected from industrial food equipment surfaces. Additional examples of environmental samples are provided below.

Helicase: The term “helicase” refers herein to an enzyme capable of unwinding a double-stranded nucleic acid enzymatically. For example, helicases are enzymes that are found in all organisms and in all processes that involve nucleic acids such as replication, recombination, repair, transcription, translation and RNA splicing. Helicases use the energy of nucleoside triphosphate (for example ATP) hydrolysis to break the hydrogen bonds that hold the strands together in duplex DNA and RNA. A helicase may translocate along DNA or RNA in a 5′ to 3′ direction or in the opposite 3′ to 5′ direction. Helicases can be found in prokaryotes, viruses, archaea, and eukaryotes or recombinant forms of naturally occurring enzymes as well as analogues or derivatives having the specified activity. Examples of naturally occurring DNA helicases, described by Kornberg and Baker in chapter 11 of their book, DNA Replication, W. H. Freeman and Company (2^{nd} ed. (1992)), include E. coli helicase I, II, III, & IV, Rep, DnaB, PriA, PcrA, T4 Gp41 helicase, T4 Dda helicase, T7 Gp4 helicases, SV40 Large T antigen, yeast RAD. Additional helicases include RecQ helicase (Harmon and Kowalczykowski, J. Biol. Chem. 276:232-243 (2001)), thermostable UvrD helicases from T. tengcongensis and T. thermophilus (Collins and McCarthy, Extremophiles. 7:35-41. (2003)), thermostable DnaB helicase from T. aquaticus (Kaplan and Steitz, J. Biol. Chem. 274:6889-6897 (1999)), and MCM helicase from archaeal and eukaryotic organisms (Grainge et al., Nucleic Acids Res. 31:4888-4898 (2003)).

Helicase-dependent amplification: The term “helicase-dependent amplification” or “HDA” refers to an in vitro method for amplifying nucleic acids by using a helicase for unwinding a double-stranded nucleic acid to generate templates for primer hybridization and subsequent primer-extension. This process utilizes two oligonucleotide primers, each hybridizing to the 3′-end of either the sense strand containing the target sequence or the anti-sense strand containing the reverse complementary target sequence. The HDA reaction is a general method for helicase-dependent nucleic acid amplification.

Hybridization: The term “hybridization” refers to binding of a single-stranded nucleic acid to a complementary single-stranded nucleic acid, preferably under conditions in which binding occurs only specifically to a nucleic acid region having a complementary sequence and not to other regions. In some embodiments, hybridization occurs between an oligonucleotide and a complementary region of a single-stranded nucleic acid. The specificity of hybridization may be influenced by the length of the oligonucleotide, the temperature in which the hybridization reaction is performed, the ionic strength, and the pH. In some embodiments, hybridization occurs between a primer and a complementary single-stranded region on a target nucleic acid to facilitate polymerase-dependent replication of the target nucleic acid and/or reverse-transcriptase-dependent synthesis of cDNA from a target nucleic acid serving as an RNA template. In some embodiments, hybridization occurs between a probe and a nucleic acid of interest. In some embodiments, hybridization refers to binding of an oligonucleotide primer to a region of the single-stranded nucleic acid template under conditions in which the primer binds only specifically to its complementary sequence on one of the template strands, not other regions in the template. In some embodiments, hybridization occurs between two single-stranded nucleic acids that are at least 95%, at least 96%, at least 97%, at least 98%, at least 99% or 100% complementary.

Hybridization specificity: As used herein, the term “hybridization specificity” refers to the ability of a molecule comprising or consisting of a single-stranded polynucleotide, or a portion of said molecule, to anneal to a complementary region of a polynucleotide. The degree of hybridization may vary depending on conditions (such as temperature, pH, buffers, etc.) and depending on level of complementarity. In some embodiments, a molecule or portion thereof, may have hybridization specificity for a region of a polynucleotide that is at least 95%, at least 96%, at least 97%, at least 98%, at least 99% or 100% complementary to the stated molecule or portion thereof.

Identity: The term “identity” as known in the art, refers to a relationship between two or more sequences, as determined by comparing the sequences. In the art, identity also means the degree of sequence relatedness between sequences, as determined by the number of matches between strings of two or more residues (amino acid or nucleic acid). Identity measures the percent of identical matches between two or more sequences with gap alignments (if any) addressed by a particular mathematical model or computer program (i.e., “algorithms”). Identity of related sequences can be readily calculated by known methods. Percent identity may be determined, for example, by comparing sequence information using sequence alignment programs known to those skilled in the art.

Incubation: The term “incubation” refers to the process of exposing something (e.g., a sample) to a set of conditions (e.g, temperature, specific reagents, etc.) for a period of time. As used herein, a “prolonged incubation” refers to incubation for a period of time that is longer than at least one hour or at least two hours.

Isothermal: The term “isothermal” used in the context of helicase-dependent amplification refers to nucleic acid amplification that occurs at a constant temperature.

Lysis: As used herein, “lyse” or “lysis” refers to breaking open cells (e.g., bacterial cells) to release its contents (e.g., nucleic acids). Lysis may be achieved by contacting a sample comprising cells with a lysis buffer comprising lytic components. Examples of lytic components may be, but are not limited to, detergents, enzymes or denaturing salts. A fluid comprising the contents of lysed cells is referred to as a “lysate.”

Lytic enzyme: As used herein, the term “lytic enzyme” refers to any enzyme that promotes lysis of a cell. Preferably, the cell is a bacterial cell. For example, lytic enzymes promote lysis of a bacterial cell by hydrolyzing the bacterial cell wall. Non-limiting examples of lytic enzymes are lysozyme and mutanolyisn.

Melting: The terms “melting”, “unwinding” or “denaturing” refer to separating all or part of two complementary strands of a nucleic acid duplex.

Nucleic acid: The terms “nucleic acid molecule,” “nucleic acid,” “oligonucleotide,” and “polynucleotide” may be used interchangeably, and refer to a polymer of nucleotides. Such polymers of nucleotides may contain natural and/or non-natural nucleotides. Illustrative nucleic acids or polynucleotides include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs) or hybrids thereof. “Nucleic acid sequence” refers to the linear sequence of nucleotides of the nucleic acid molecule or polynucleotide. In some instances, the nucleic acid may be a short molecule (approximately 13-25 nucleotides long and/or less than 200 nucleotide residues) and may then be termed an “oligonucleotide.” The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules, plasmids, supercoiled DNA and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). DNA includes, but is not limited to, cDNA, genomic DNA, plasmid DNA, synthetic DNA, and semi-synthetic DNA. Those molecules which are double stranded nucleic acid molecules may be nicked or intact. The double stranded or single-stranded nucleic acid molecules may be linear or circular. The duplexes may be blunt ended or have single-stranded tails. The term “duplex” refers to a nucleic acid molecule that is double-stranded in whole or part. The single-stranded molecules may have secondary structure in the form of hairpins or loops and stems. Nucleic acids may be isolated, cloned or synthesized in vitro by means of chemical synthesis. Any of the above described nucleic acids may be subject to modification where individual nucleotides within the nucleic acid are chemically altered (for example, by methylation). Modifications may arise naturally or by in vitro synthesis.

Presence or absence: The phrase “presence or absence” when used in reference to detecting presence or absence of amplified nucleic acid or determining presence or absence of a target bacteria, refers to the state of having or not having the stated composition (e.g., amplified nucleic acid or target bacteria). The presence and absence assay signal rely on the limit of detection of the assay. As used herein, “absence” does not necessarily mean absolute absence; instead, a composition may be determined as absent if there is a low level, such as below a negative control threshold, that is detected or if the concentration of the composition in a test sample is close to the limit of detection or much lower than the limit of detection of the assay. As used herein, “presence” may be determined by a specific cut-off, below which the composition is not considered to be present. For samples with concentrations close to the limit of detection, multiple replicates may be necessary to detect presence.

Primer: As used herein, the term “primer” refers to a single-stranded nucleic acid capable of binding to a single-stranded region on a target nucleic acid to facilitate polymerase-dependent replication of the target nucleic acid and/or reverse-transcriptase-dependent synthesis of cDNA from a target nucleic acid serving as an RNA template. In some embodiments, a primer is capable of binding to a region on an RNA molecule. In some embodiments, a primer is capable of binding to a single-stranded region on a DNA or cDNA molecule. In some embodiments, a primer is capable of binding to both a single-stranded region on an RNA molecule and a single-stranded region on a DNA or cDNA molecule. The term “primer pair” refers to a set of two primers, one serving as the forward primer and the other as the reverse primer, each binding to one of the two ends of a single-stranded region on a target nucleic acid. A primer of the present disclosure generally has less than 50 residues. Preferably, a primer of the present disclosure is in a size range having a lower limit of about 5 to about 15 residues and an upper limit of about 25 to about 35 residues. For example, a primer of the present disclosure may comprise 14, 15, 16, 17, 18, 19, 20, 21, 22, 23, 24, 25, 26, 27, 28, or 29 residues.

Probe: As used herein, the term “probe” refers to a labeled molecule or portion thereof that is designed to detect a nucleic acid of interest. In some embodiments, the term “probe” is interchangeable with the term “molecular probe.” When used in reference to a probe, the term “detect” is interchangeable with the term “recognize” and refers to the ability of the probe to identify the nucleic acid of interest. In some embodiments, a probe is a single-stranded nucleic acid comprising one or more complementary sequences to the nucleic acid of interest. When the probe is placed in contact with a sample under conditions that allow the probe to hybridize with the nucleic acid of interest, the nucleic acid of interest is detected. The label of the probe can be a tag, such as a radioactive or chemical tag, that allows hybridization of the probe to the nucleic acid of interest to be visualized. In some embodiments, the probe is a fluorescent molecular probe (also referred to herein as a “fluorescent probe”), which is a probe that emits fluorescence. An example of a fluorescent molecular probe is a conditional fluorescent hybridization probe. In some embodiments, the probe is a conditional fluorescent hybridization probe that emits fluorescence when hybridized to the nucleic acid of interest. In some embodiments, the nucleic acid of interest is an amplicon produced by helicase-dependent amplification (HDA) according to methods of the present disclosure.

Salmonella: As used herein, the term “Salmonella” refers to all species of the gram-negative rod-shaped bacteria in the genus of Salmonella, including S. bongori, S. enterica, S. enterica subsp. arizonae, S. enterica subsp. diarizonae, S. enterica subsp. enterica, S. enterica subsp. houtenae, S. enterica subsp. indica, and S. enterica subsp. salamae. “Salmonella” also refers to all serotypes, including, for example, S. typhimurium, S. blockley, and S. reading.

Sensitivity: The term “sensitivity” when used in reference to a detection method or assay is the proportion of actual positive samples that are correctly identified as positive by the method or assay. Actual positive samples are generally defined by using a validated assay used to detect the presence of a target microbe (e.g., target bacteria). In some embodiments the actual positives are defined by a method comprising culturing a sample to determine whether target microbes are present. In some embodiments, one or more positive control samples are used as actual positive samples. In some embodiments, one or more positive control samples are used alongside test samples that may or may not be actual positive samples. In some embodiments, a method is described as having “sufficient sensitivity,” which refers to the ability of the method to correctly identify as positive an actual positive sample. In some embodiments, a method may have sufficient sensitivity under certain conditions.

Specificity: The term “specificity” when used in reference to a detection method or assay is the proportion of actual negative samples that are correctly identified as negative by the method or assay. Actual negative samples are generally defined by using a validated assay used to detect the presence of a target microbe (e.g., target bacteria). In some embodiments the actual negatives are defined by a method comprising culturing a sample to determine whether target microbes are present. In some embodiments, one or more negative control samples are used as actual negative samples. In some embodiments, one or more negative control samples are used alongside test samples that may or may not be actual negative samples. In some embodiments, a negative control sample comprises a bacterium that is not the target bacterium.

Target bacteria: The term “target bacteria,” as used herein, refers to one or more species of bacteria that are targeted for detection and/or quantification in a sample, such as an environmental sample. In some embodiments, the target bacteria are any bacteria of the genus Salmonella.

Target nucleic acid: According to the present disclosure, a “target nucleic acid,” refers to a nucleic acid molecule, or portion thereof, that is present in target bacteria. In some embodiments the target nucleic acid is detected using nucleic acid detection methods. Methods for detecting a target nucleic acid may be used for purposes of determining the presence or absence of the target bacteria in a sample. In some embodiments, a target nucleic acid is detected using the compositions and methods provided by the present disclosure. In some embodiments, a target nucleic acid is detected using methods comprising helicase-dependent amplification (HDA). In such cases, the target nucleic acid is amplified according to HDA methods and is referred to as an HDA target nucleic acid. Specifically, the term “HDA target nucleic acid” refers to a whole or part of nucleic acid to be selectively amplified and which is defined by 3′ and 5′ boundaries. The HDA target nucleic acid may also be referred to as a fragment or sequence that is intended to be amplified. The size of the HDA target nucleic acid to be amplified may be, for example, in the range of about 50 base pairs (bp) to about 5000 bp. In preferred embodiments, the size of the HDA target nucleic acid to be amplified is 50-150 bp.

The HDA target nucleic acid may be contained within a longer double-stranded or single-stranded nucleic acid. Alternatively, the HDA target nucleic acid may be an entire double-stranded or single-stranded nucleic acid. If the initial nucleic acid provided for an HDA method is RNA, the RNA (or a region of the RNA) is reverse transcribed into a cDNA molecule and the cDNA is amplified by a DNA polymerase. Although the cDNA is being amplified in this scenario, the HDA target nucleic acid is considered to be the initial RNA because the RNA is present in target bacteria and the reverse-transcribed copy of the RNA (i.e., the cDNA) is what is being amplified by HDA.

### Embodiments

In the following description, numerous specific details are given to provide a thorough understanding of the embodiments. The embodiments can be practiced without one or more of the specific details, or with other methods, components, materials, etc. In other instances, well-known structures, materials, or operations are not shown or described in detail to avoid obscuring aspects of the embodiments.

Reference throughout this specification to “one embodiment,” “an embodiment,” or “embodiments” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Unless indicated otherwise, when a range of any type is disclosed or claimed, it is intended to disclose or claim individually each possible number that such a range could reasonably encompass, including any sub-ranges encompassed therein. Moreover, when a range of values is disclosed or claimed, which Applicant intends to reflect individually each possible number that such a range could reasonably encompass, Applicant also intends for the disclosure of a range to reflect, and be interchangeable with, disclosing any and all sub-ranges and combinations of sub-ranges encompassed therein. Where ranges are given, endpoints are included.

Salmonella Contamination and Environmental Detection

Salmonella is a genus of gram-negative bacteria comprising two recognized species, Salmonella enterica (S. enterica) and Salmonella bongori (S. bongori). S. enterica is divided into six subspecies: S. enterica subsp. arizonae, S. enterica subsp. diarizonae, S. enterica subsp. enterica, S. enterica subsp. houtenae, S. enterica subsp. indica, and S. enterica subsp. salamae. The abbreviation Salmonella spp. is sometimes used in the art in place of the phrase “Salmonella species,” and may refer to all species and subspecies of Salmonella or to specific species or subspecies, depending on the context.

The species S. enterica has over 2500 known serovars (also referred to as “serogroups” or “serotypes”). Serovars are classified based on antigens presented by these organisms. Antibodies for particular antigens can be used to serotype bacteria.

Salmonella species can cause illness. Two main groups of Salmonella serotypes known to cause illness are the typhoidal serotypes and nontypohoidal serotypes. Both typhoidal and nontyphoidal infection can cause salmonellosis, a symptomatic infection with common symptoms including diarrhea, fever, abdominal cramps, and vomiting. In some cases, Salmonella infection can be fatal.

Nontyphoidal serotypes can be transferred from animal-to-human and from human-to-human. Infection can occur when a person ingests foods that contain these bacteria. Nontyphoidal serotypes can cause bloodstream infections with varied symptoms including fever, hepatosplenomegaly, and respiratory symptoms. Nontyphoidal serotypes include Salmonella enterica serotype Typhimurium, Salmonella enterica serotype Enteritidis, and Salmonella enterica serotype Blockley. Typhoidal Salmonella serotypes can be transferred from human-to-human. Typhoidal serotypes can cause food-borne infection, typhoid fever and paratyphoid fever. Typhoidal serotypes include Salmonella enterica serotype Typhi and Salmonella enterica serotype Paratyphi A.

Most Salmonella infections are caused by food or water contaminated with the bacteria. Contamination can occur when food or water comes in contact with feces of infected people or animals. A variety of foods can be the source of Salmonella infection. Examples of foods that have the potential to be contaminated with Salmonella include sprouts and other vegetables, eggs, chicken, pork, fruits, and processed foods. Recently (between 2017-2019), human outbreaks of Salmonella enterica serotype Reading in the U.S. and Canada have been linked to the consumption of raw turkey products. Groups that are most susceptible to infection include children, pregnant women, elderly people, and those with weakened immune systems.

Traditional methods for detection of Salmonella in environmental samples include environmental sample collection followed by enrichment through growth media and finally detection by one of many schemes such as, but not limited to, the following: selective/differential media agar, antibody-based assays (e.g., ELISA and lateral flow assays), molecular methods such as DNA/RNA amplification (e.g., PCR, LAMP, and NEAR) or hybridization assays with nucleic acid probes. For example, traditional methods for Salmonella detection can involve procedures using serial enrichments with increasing selectivity culminating in the isolation of Salmonella on selective-differential agar plates. More recently, PCR and real-time, quantitative PCR (qPCR) have been used for detection in food and environmental samples.

The present disclosure provides methods for rapid detection of Salmonella from environmental samples via detection of Salmonella target nucleic acids. In various embodiments, the methods comprise the steps of providing an environmental sample to be tested; contacting an aliquot of the environmental sample with a lysis mixture under conditions to lyse at least a portion of cells in the aliquot, thereby generating a lysate; contacting an aliquot of the lysate with a detection mixture, thereby generating an assay mixture; in the assay mixture, reverse transcribing target RNA to form target cDNA and amplifying the target cDNA by helicase-dependent amplification (HDA); and detecting presence or absence of the amplified target cDNA, to thereby determine the presence or absence of Salmonella in the environmental sample. In some embodiments, the provided methods further comprise a step of pre-treating the aliquot of the environmental sample to remove nucleic acids not associated with intact cells prior to contacting the aliquot with the lysis mixture.

### Environmental Samples

In various embodiments, a sample is provided for purposes of testing for the presence or absence of target bacteria in the sample. The sample can be any sample that may comprise target bacteria. In some embodiments, the provided sample is suspected of being contaminated with bacteria, for example, the target bacteria. In some embodiments, providing a sample to be tested comprises providing a sample to confirm absence of bacterial contamination, for example, absence of the target bacteria. In some embodiments, the target bacteria are of the genus Salmonella.

In some embodiments, the presence or absence of target bacteria can be analyzed in a test environmental sample that is derived from food processing and/or beverage processing environmental sources. Non-limiting examples of food processing and/or beverage processing environmental sources include food-handling surface samples (e.g., conveyor belts, blades, cutting surfaces, mixing equipment surfaces, filters, storage containers), room samples (e.g., walls, floors, drains, ventilation equipment), and cleaning equipment (e.g., hoses, cleaning tools).

In preferred embodiments, the sample is an environmental sample. In preferred embodiments, an environmental sample is collected from the environment in a room or a building where foods and/or beverages are produced and/or processed. In some preferred embodiments, environmental samples may be collected from food/beverage production, processing or service sites.

In some embodiments, the environmental sample is from an environment comprising a low concentration of target bacteria cells (e.g., Salmonella cells). As used herein, a “low concentration” of target bacteria cells refers to a concentration that is difficult to detect without amplifying the amount of an indicator associated with the target bacteria (such as by amplifying the amount of cells or the amount of target bacterial nucleic acids). For example, while it is expected that certain environments from food/beverage production, processing or service sites contain no target bacteria like Salmonella, contamination may result in a low concentration of the target bacteria at these sites. Therefore, in some embodiments, the environmental sample is from an environment that can be tested for bacterial contamination of the target bacteria.

In some embodiments, the environment is a surface that is a solid comprising a low concentration of target bacteria cells. In some embodiments, a low concentration of target bacteria is at most 200 colony forming units (CFU) per 1 square inch of a solid surface. In some embodiments, a low concentration of target bacteria is at most 100 CFU per 1 square inch of a solid surface. In some embodiments, a low concentration of target bacteria is at most 50 CFU per 1 square inch of a solid surface. In some embodiments, a low concentration of target bacteria is at most 5 CFU per 1 square inch of a solid surface. In some embodiments, a low concentration of target bacteria is at most 200 CFU per 1 milliliter of a collected sample. In some embodiments, a low concentration of target bacteria is at most 100 CFU per 1 milliliter of a collected sample. In some embodiments, a low concentration of target bacteria is at most 50 CFU per 1 milliliter of a collected sample. In some embodiments, a low concentration of target bacteria is at most 5 CFU per 1 milliliter of a collected sample. In some embodiments, the solid surface comprises from about 10 to about 200 colony forming units (CFU) of target bacteria per 1 square inch of the solid surface. In some embodiments, the solid surface comprises from about 10 to about 100 CFU of target bacteria per 1 square inch of the solid surface. In some embodiments, the solid surface comprises from about 10 to about 50 CFU of target bacteria per 1 square inch of the solid surface. In some embodiments, the solid surface comprises from about 5 to about 200 colony forming units (CFU) of target bacteria per 1 square inch of the solid surface. In some embodiments, the solid surface comprises from about 5 to about 100 CFU of target bacteria per 1 square inch of the solid surface. In some embodiments, the solid surface comprises from about 5 to about 50 CFU of target bacteria per 1 square inch of the solid surface. In some embodiments, the target bacteria are of the genus Salmonella.

In various embodiments, samples, such as environmental samples, may be collected with a collection device. In some embodiments, a collection device collects an environmental sample from a surface.

In some embodiments, the collection device is a swab. The swab may be made from various materials, such as, but not limited to, cotton, polyester or polyurethane. In some embodiments, the swab is pre-moistened, such as in solution. The swab may be pre-moistened in buffer or broth. In some embodiments, the swab is pre-moistened in neutralizing buffer, buffered peptone water, or culture medium. In some embodiments, the volume of the pre-moistening liquid on the swab is about 1-10 mL.

In some embodiments, the collection device is a sponge. The sponge may be made from various materials, such as, but not limited to, polyurethane. In some embodiments, a sponge advantageously samples a larger surface area than other sampling devices. In some embodiments, the sponge is pre-moistened. The sponge may be pre-moistened in buffer or broth. In some embodiments, the sponge is pre-moistened in neutralizing buffer, buffered peptone water, or culture medium. In some embodiments, the volume of the pre-moistening liquid on the sponge is about 10-25 mL.

In some embodiments, a sample collection device (e.g., a swab, a sponge) containing sample material may be used to provide an environmental sample according to methods of the present disclosure. In some embodiments, the sample material may be eluted (e.g., rinsed, scraped, expressed) from a sample collection device before using the sample material in a method of the disclosure. In some embodiments, liquid or solid samples may be diluted in a liquid (e.g., water, buffer, broth).

In some embodiments, collecting an environmental sample comprises swabbing with the collection device (e.g., swab or sponge) an area of a solid surface. In some embodiments, the area is about a 1×1 inch area, a 4×4 inch area or a 12×12 inch area. In some embodiments, swabbing comprises swabbing in multiple directions. In some embodiments, the surface is a flat surface. In some embodiments, the environmental sample collected comprises from about 5 to about 200 CFU of target bacteria (e.g., Salmonella). In some embodiments, the environmental sample collected comprises from about 5 to about 100 CFU of target bacteria (e.g., Salmonella). In some embodiments, the environmental sample collected comprises from about 5 to about 50 CFU of target bacteria (e.g., Salmonella). In some embodiments, the environmental sample collected comprises from about 10 to about 200 CFU of target bacteria (e.g., Salmonella).

Methods and kits of the present invention may include a collection device.

### Target Nucleic Acids for Detection of Salmonella

Provided by the present disclosure are compositions and methods for detection of target nucleic acids found in target bacteria cells. In various aspects, the target bacteria are Salmonella.

A target nucleic acid may be any type of nucleic acid molecule, such as ribonucleic acid (RNA) or deoxyribonucleic acid (DNA). In some embodiments, the target nucleic acid is an RNA, and is also referred to herein as a “target RNA.”

In some embodiments, a target nucleic acid is abundant in target bacteria, thereby lowering the limit of detection of assays such as those disclosed herein. In some embodiments, a target nucleic acid is present at any or all stages of growth for the target bacteria. For example, the target nucleic acid is present in stationary phase and log phase growth. In some embodiments, the target bacteria are of the genus Salmonella. In some embodiments, a target nucleic acid is present at all stages of growth for Salmonella.

In various embodiments, a target nucleic acid of the present disclosure comprises a nucleic acid sequence from bacterial 23S ribosomal RNA (rRNA). The 23S rRNA is a component of the large subunit of the bacterial ribosome. In some embodiments, a target nucleic acid comprises a sequence from Salmonella 23S rRNA. The 23S rRNA is an RNA molecule present in Salmonella at a high copy number.

The 23S rRNA is a highly folded RNA molecule, making some regions inaccessible if they are buried within the folds. Inaccessibility of the target nucleic acid can compromise detection. A target nucleic acid that is inaccessible to detection agents (such as enzymes and primers) may not be easily detected. In some embodiments, the target nucleic acid of the present disclosure, or a region thereof, is accessible to detection methods, such as those disclosed herein.

In some embodiments, a target nucleic acid of the present disclosure demonstrates specificity for the target bacteria, for example, Salmonella. In some embodiments, the target nucleic acid is a region in the 23S rRNA that is specific to Salmonella enterica serovars and is not present in the closely related Escherichia coli.

In some embodiments, the target RNA comprises the nucleic acid sequence (read in the 5′ to 3′ direction) of GAG AAG GCA CGC UGA CAC GUA GGU GAA GUG AUU UAC UCA CGG AGC UGA AGU CAG (SEQ ID NO: 1). In some embodiments, the target RNA comprises a nucleic acid sequence having at least 90%, 91%, 92%, 93%, 94%, 95%, 96%, 97%, 98%, 99%, or 100% sequence identity to SEQ ID NO: 1.

An advantageous property of the target nucleic acids provided by the present disclosure is specificity for the bacteria of interest (e.g., Salmonella), as demonstrated by examples provided herein. Additionally, the present disclosure demonstrates that these target nucleic acids are accessible for detection, for example, by using compositions and methods provided herein.

Forward and reverse primers as well as probes for the target nucleic acid of the present disclosure can be designed according to known molecular biology techniques. For example, primers according to the present disclosure can be used to reverse transcribe a target RNA to form a corresponding target cDNA. As used herein, a “corresponding target cDNA” or simply “target cDNA” is a cDNA that is generated by reverse transcribing a target RNA. Additionally, in some embodiments, primers can be used to amplify a target cDNA. In some embodiments, a single pair of one forward primer and one reverse primer can be used to reverse transcribe a target RNA to form a corresponding target cDNA and to amplify the target cDNA.

In some embodiments, Salmonella detection may include use of a first primer comprising the nucleic acid sequence of 5′ GAG AAG GCA CGC TGA CAC 3′(SEQ ID NO: 2) and a second primer comprising the nucleic acid sequence of 5′ CTG ACT TCA GCT CCG TGA GTA AAT 3′ (SEQ ID NO: 3).

Also provided herein is a primer comprising or consisting of the nucleic acid sequence of GAG AAG GCA CGC TGA CAC (SEQ ID NO: 2) or a sequence with at least 90%, at least 95%, at least 96%, at least 97%, at least 98% or at least 99% sequence identity to SEQ ID NO: 2. Also provided herein is a primer comprising or consisting of the nucleic acid sequence of CTG ACT TCA GCT CCG TGA GTA AAT (SEQ ID NO: 3) or a sequence with at least 90%, at least 95%, at least 96%, at least 97%, at least 98% or at least 99% sequence identity to SEQ ID NO: 3. Also provided are compositions and kits comprising one or more of any of these primers.

### Removing Nucleic Acids not associated with Intact Cells

False positives from lysed cells in the environment has been a problem with previous environmental sampling procedures. For example, methods that rely on detection of target nucleic acids may inadvertently detect nucleic acids from lysed cells existing in the environment. Nucleic acids from lysed cells are also referred to by the present disclosure as “free nucleic acids,” because they have been released from ruptured cells in the environment, such as dead, dying, lysed, or lysing cells in the environment.

The present disclosure provides compositions and methods for removing nucleic acids from lysed cells prior to downstream methods, including downstream methods of detecting target nucleic acids from intact cells in an environmental sample. The present disclosure further provides compositions and methods to easily and effectively deactivate reactions that remove nucleic acids from lysed cells so as not to remove nucleic acids from intact, living cells. In some embodiments, the provided methods to remove nucleic acids from lysed cells are referred to herein as “pre-treatment.”

Pre-treatment comprises use of one or more enzymes that cleave nucleic acids. For example, pre-treatment cleaves DNA and RNA, as well as other single-stranded and double-stranded nucleic acids. The one or more enzymes for pre-treatment may comprise an RNase, a DNase, and/or an enzyme that functions both as an RNase and a DNase. In some embodiments, an enzyme for pre-treatment is micrococcal nuclease from Staphylococcus aureus. Methods of the present disclosure also comprise a mechanism to inactivate the one or more enzymes so that nucleic acids from living cells are essentially not cleaved by the enzyme. As used herein, the phrase “essentially not cleaved” means that the nucleic acids from living cells are not cleaved or only minimally cleaved.

In some embodiments, the present disclosure provides for a sample pre-treatment to remove nucleic acids from lysed cells prior to lysing intact bacterial cells collected in a sample and other downstream methods. In some embodiments, pre-treatment involves a reaction that is active prior to the cell lysis method step of the present disclosure, but inactivated before lysis occurs. In some embodiments, the pre-treatment is applied prior to cell lysis and helicase-dependent amplification (HDA) methods.

In some embodiments, compositions and methods of the present disclosure incorporate a pre-treatment for removing nucleic acids from samples that are not associated with intact Salmonella cells.

Pre-treatment according to the present disclosure comprises use of a pre-treatment mixture comprising pre-treatment components. In some embodiments, the set of pre-treatment components comprises an enzyme that cleaves nucleic acids and a divalent salt required for the enzyme's activity. In some embodiments, the mechanism or component to inactivate the enzyme functions by making the divalent salt inaccessible to the enzyme. For example, a divalent ion chelator may be used to inactivate pre-treatment enzyme activity. Examples of divalent ion chelators include the following: ethylenediaminetetraacetic acid (EDTA); BAPTA (1,2-bis(o-aminophenoxy)ethane-N,N,N′,N′-tetraacetic acid) and its derivatives (e.g., BAPTA-AM); EGTA derivatives (e.g., EGTA-AM); citric acid; FURA 2 and its derivatives; RHOD 2 and its derivatives; and FLUO 3 and its derivatives.

An example of an enzyme that may be used as a component of pre-treatment is micrococcal nuclease. An example of a divalent salt required for the enzyme's activity is calcium chloride (CaCl₂). An example of a mechanism to inactive micrococcal nuclease activity by making the divalent salt inaccessible is contacting a pre-treated sample with egtazic acid (EGTA). EGTA functions as a calcium chelator.

In some embodiments, the set of pre-treatment components may further comprise a pH buffer to produce an optimal pH for the enzyme. An example of a pH buffer is Tris buffer.

In some embodiments, the set of pre-treatment components may further comprise one or more components that improve stability of the enzyme in storage. For example, bovine serum albumin (BSA) can be included as a component of a pre-treatment mixture that improves stability of micrococcal nuclease in storage.

In some embodiments, the set of pre-treatment mixture components comprises one or more of the components listed in Table 1. In some embodiments, the set of pre-treatment mixture components comprises the components listed in Table 1.

In some embodiments, the set of pre-treatment mixture components comprises micrococcal nuclease and calcium chloride (CaCl₂). In some embodiments, the set of pre-treatment mixture components comprises one or more of: (i) micrococcal nuclease, (ii) calcium chloride (CaCl₂); (iii) Tris buffer (e.g., Tris-HCl pH 8.8); and (iv) BSA. In some embodiments, the set of pre-treatment mixture components comprises (i) micrococcal nuclease, (ii) calcium chloride (CaCl₂); (iii) Tris buffer (e.g., Tris-HCl pH 8.8); and (iv) BSA. In some embodiments, the pre-treatment mixture further comprises sucrose. In some embodiments, the pre-treatment mixture further comprises dextran.

In some embodiments, the pre-treatment mixture is lyophilized. In some embodiments, a lyophilized pre-treatment mixture is resuspended. For example, a lyophilized pre-treatment mixture may be resuspended with a liquid composition. For example, the lyophilized pre-treatment mixture may be resuspended with a sample or solution comprising target bacteria cells. In some embodiments, the concentration of one or more of the components of the pre-treatment mixture after resuspension is in the range of concentrations listed in Table 1. In some embodiments, the set of pre-treatment mixture components comprises the components listed in Table 1 and the concentration of each of the listed components after resuspension of the lyophilized pre-treatment mixture is in the range specified in Table 1. In some embodiments, the set of pre-treatment mixture components comprises one or more of the components listed in Table 2 and the concentration of each component after resuspension of the lyophilized pre-treatment mixture is the concentration listed in Table 2. In some embodiments, the set of pre-treatment mixture components comprises the components listed in Table 2 and the concentration of each of the listed components after resuspension of the lyophilized pre-treatment mixture is the concentration listed in Table 2.

TABLE 1
Concentration ranges for pre-treatment mixture components

| Component | Concentration range | |
|---|---|---|
| Tris HCl pH 8.8 | 5-50 | mM |
| CaCl2 | 2-6 | mM |
| Micrococcal Nuclease | 0.1-0.3 | Units/μL |
| BSA | 0.25-0.8 | mg/mL |
| Sucrose | 5-7.5% | (m/v) |
| Dextran | 1-2% | (m/v) |

TABLE 2
Pre-treatment mixture components and concentrations

| Component | Concentration | |
|---|---|---|
| Tris HCl pH 8.8 | 10.6 | mM |
| CaCl2 | 4.1 | mM |
| Micrococcal Nuclease | 0.22 | Units/μL |
| BSA | 0.52 | mg/mL |
| Sucrose | 6.86% | (m/v) |
| Dextran | 1.29% | (m/v) |

In some embodiments, the mechanism to inactivate micrococcal nuclease is addition of EGTA, a calcium chelator. The EGTA binds to calcium with high affinity, removing it from solution and making it inaccessible to micrococcal nuclease. Once EGTA is added, micrococcal nuclease can no longer cleave nucleic acids. This prevents the enzyme from removing potential target nucleic acids from intact cells collected in a sample.

In some embodiments, the enzyme in the set of pre-treatment components cleaves nucleic acids at a temperature matching the lysis temperature of the cells in a sample. Such property of the enzyme will enable improved workflow efficiency for sample analysis.

In some embodiments, a method of the present disclosure comprises contacting a provided sample or aliquot thereof with a pre-treatment mixture under conditions to remove nucleic acids not associated with intact cells in the sample or aliquot of the sample.

### Bacterial Cell Lysis

Methods of the present disclosure include cell lysis to release molecules, including nucleic acids such as DNA and RNA, from cells. Following release of nucleic acids, additional methods may be employed for processing the nucleic acids, including reverse transcription, amplification and/or detection of nucleic acids. The provided lysis methods include advantages over other lysis methods, such advantages including ease of use, lytic efficiency and non-inhibitory properties to downstream methods such as helicase-dependent amplification (HDA).

Provided herein are compositions and methods to lyse bacterial cells. These compositions and methods may be used for lysing gram-negative bacteria and/or gram-positive bacteria. In some embodiments, cell lysis compositions and methods of the disclosure are used for lysing gram-negative bacteria, such as Salmonella. In some embodiments, cell lysis compositions and methods of the disclosure are used for lysing gram-positive bacteria, such as Listeria. In some embodiments, cell lysis compositions and methods of the disclosure are used for lysing both gram-negative bacteria and gram-positive bacteria.

Gram-negative bacteria, such as Salmonella, can be lysed by only heating (e.g., to approximately 65° C.-80° C.). For example, Salmonella can be lysed with a water or buffer solution and heating to approximately 80° C. for approximately 20 minutes. However, other components are needed to lyse gram-positive bacteria. Lysis components described by the present disclosure can be used to lyse both gram-negative bacteria and gram-positive bacteria.

Cell lysis according to the present disclosure comprises use of a lysis mixture comprising lysis mixture components. In some embodiments, the set of lysis mixture components comprises: (i) at least one lytic enzyme (for example, lysozyme and/or mutanolysin); (ii) at least one protease or enzyme that degrades protein (for example, proteinase K and/or achromopeptidase); (iii) a chelating resin; and (iv) a pH buffer (for example, Tris). In some embodiments, the chelating resin is Chelex®-100 resin (also referred to herein as simply “Chelex-100” or “Chelex”), which is identifiable by CAS number 11139-85-8. Chelex-100 is a styrene divinylbenzene copolymer containing paired iminodiacetate ions. Chelex-100 is an insoluble resin that chelates metals and divalent cations, and is known for lysing bacterial cells. In some embodiments, the pH buffer should be selected to produce an optimal pH for a downstream HDA method. In some embodiments, an optimal pH is a pH of about 8.8. While these components can be used to lyse gram-negative bacteria such as Salmonella, they are not necessary for Salmonella lysis, since as disclosed above, gram-negative bacteria can be lysed by heating to approximately 80° C. These components do, however, enable lysis of gram-positive bacteria. Thus, the same lysis mixture described by the present disclosure can be used for gram-negative and gram-positive bacteria.

In some embodiments, cell lysis described herein occurs after pre-treatment, such as the pre-treatment methods described above. In some embodiments, the set of lysis mixture components comprises a component to inactivate pre-treatment. In some embodiments, a component to inactivate pre-treatment is a divalent ion chelator. Examples of divalent ion chelators include the following: EDTA, BAPTA and its derivatives (e.g., BAPTA-AM), EGTA derivatives (e.g., EGTA-AM), citric acid, FURA 2 and its derivatives, RHOD 2 and its derivatives, and FLUO 3 and its derivatives. For example, as described above, EGTA can inactivate pre-treatment.

In some embodiments, the set of lysis mixture components comprises one or more of: (i) lyosozyme; (ii) mutanolysin; (iii) proteinase K; (iv) Chelex®-100; (v) Tris buffer; and (vi) EGTA. In some embodiments, the set of lysis mixture components comprises: (i) lyosozyme; (ii) mutanolysin; (iii) proteinase K; (iv) Chelex®-100; (v) Tris buffer; and (vi) EGTA. In some embodiments, the set of lysis mixture components further comprises sucrose. In some embodiments, the set of lysis mixture components further comprises dextran.

In some embodiments, the set of lysis mixture components comprises one or more of: (i) lyosozyme; (ii) mutanolysin; (iii) proteinase K; (iv) achromopeptidase; (v) Chelex®-100; (vi) Tris buffer; and (vii) EGTA. In some embodiments, the set of lysis mixture components comprises: (i) lyosozyme; (ii) mutanolysin; (iii) proteinase K; (iv) achromopeptidase; (v) Chelex®-100; (vi) Tris buffer; and (vii) EGTA. In some embodiments, the set of lysis mixture components further comprises sucrose. In some embodiments, the set of lysis mixture components further comprises dextran.

In some embodiments, the lysis mixture is lyophilized. In some embodiments, one or more components of the lysis mixture are lyophilized together as one lyophilized pellet. In some embodiments, one or more components of the lysis mixture are lyophilized separately as more than one lyophilized pellet. For example, a first set of lysis mixture components comprising at least one lytic enzyme (for example, lysozyme and/or mutanolysin) and at least one protease or enzyme that degrades protein (for example, proteinase K and/or achromopeptidase) may be lyophilized as a first pellet and a second set of lysis mixture components comprising a chelating resin (for example, Chelex-100) may be lyophilized as a second pellet.

In some embodiments, one or more lyophilized pellets, each comprising one or more components of a lysis mixture are resuspended. For example, the one or more lyophilized pellets may be resuspended with a liquid composition. In some embodiments, the one or more lyophilized pellets may be resuspended with a sample or solution comprising target bacteria cells. In some embodiments, the one or more lyophilized pellets may be resuspended with an aliquot of a sample that has been pre-treated according to pre-treatment methods described above. In some embodiments, the concentration of one or more of the components of the lysis mixture after resuspension is in the range of concentrations listed in Table 3. In some embodiments, the set of lysis mixture components comprises the components listed in Table 3 and the concentration of each of the listed components after resuspension of the lyophilized lysis mixture components is in the range specified in Table 3. In some embodiments, the set of lysis mixture components comprises one or more of the components listed in Table 4 and the concentration of each component after resuspension of the lyophilized lysis mixture components is the concentration listed in Table 4. In some embodiments, the set of lysis mixture components comprises the components listed in Table 4 and the concentration of each of the listed components after resuspension of the lyophilized lysis mixture components is the concentration listed in Table 4.

TABLE 3
Concentration ranges for lysis mixture components

| Component | Concentration range | |
|---|---|---|
| Tris HCl pH 8.8 | 5-50 | mM |
| Chelex-100 | 0-7.5% | (m/v) |
| Sucrose | 2-5% | (m/v) |
| Lysozyme | 0-1 | mg/mL |
| Proteinase K | 0-1 | mg/mL |
| Mutanolysin | 0-30 | Units/mL |
| EGTA | 0-5 | mM |
| Achromopeptidase | 0-150 | Units/mL |
| Dextran | 0-2% | (m/v) |

TABLE 4
Lysis mixture components and concentrations

| Component | Concentration | |
|---|---|---|
| Tris HCl pH 8.8 | 5.7 | mM |
| Chelex-100 | 4.5% | (m/v) |
| Sucrose | 3% | (m/v) |
| Lysozyme | 0.8 | mg/mL |
| Proteinase K | 0.8 | mg/mL |
| Mutanolysin | 20 | Units/mL |
| EGTA | 2.6 | mM |
| Achromopeptidase | 85.6 | Units/mL |
| Dextran | 0.56% | (m/v) |

In some embodiments, a lysis mixture may also be referred to herein as a “lysis buffer”.

In some embodiments, a method of the present disclosure comprises contacting an aliquot of a provided sample (e.g., environmental sample) with a lysis mixture (or lysis mixture components) under conditions to lyse at least a portion of cells in the aliquot, thereby generating a lysate.

In some embodiments, the entire collected sample is subjected to the lysis procedure, which is conducted in a small volume of lysis buffer.

In some embodiments, a method of the present disclosure comprises a step of pre-treating an aliquot of a provided sample (e.g., environmental sample) to remove nucleic acids not associated with intact cells, thereby generating a pre-treated aliquot, and contacting some or all of the pre-treated aliquot with the lysis mixture (or lysis mixture components) under conditions to lyse at least a portion of cells in the some or all of the pre-treated aliquot, thereby generating a lysate.

In some embodiments, the provided lysis compositions and methods can be used with downstream methods such as reverse transcription, helicase-dependent amplification (HDA), and/or nucleic acid detection methods.

In some embodiments, the present disclosure provides cell lysis compositions and methods that can be used with downstream methods for detection of target nucleic acids. In some embodiments, the provided lysis compositions and methods can be used with downstream methods for determining the presence or absence of target bacteria in an environmental sample.

In some embodiments, following cell lysis, some or a portion of a sample comprising lysed cells (i.e., the lysate) is used in a downstream method, such as HDA.

Among other things, the lysis methods of the present disclosure provide the advantages of improved limit of detection and improved sensitivity of downstream assays that measure target nucleic acid presence and/or levels as compared to other lysis methods.

Other lysis methods include lysis protocols for samples that are target-rich. For instance, other methods use clinical samples, such as throat swabs of patients, which contain many bacteria. Target-rich samples allow use of lysis methods that have low lytic efficiency, such as lysis in which only 10% of bacteria in a sample is lysed and yet still enable detection of the target with an assay due to the high amount of bacteria. By contrast, the present lysis method enables lysis of more bacterial cells. In some embodiments, lysis methods of the present disclosure enable lysis of at least 90% of cells in a sample. In some embodiments, lysis methods of the present disclosure enable lysis of nearly every cell in the sample. In some embodiments, the lysis method of the present disclosure enables a limit of detection of approximately 10-100 cells with downstream assays.

Another improvement of the present lysis methods provides for the elimination of harsh chemicals that need to be neutralized or diluted prior to a sample being added to or used for a downstream method, such as a detection assay. While other lysis methods use such harsh chemicals, the lysis method disclosed herein does not use chemicals that require neutralization or dilution prior to target detection. The present lysis method thus improves workflow by minimizing touchpoints and minimizing steps from sample collection through analysis.

### Helicase-Dependent Amplification

Helicase-dependent amplification (HDA), is a method for nucleic acid amplification that mimics an in vivo process of DNA replication, using helicase(s) to isothermally unwind nucleic acid duplexes. The resulting separated strands of the nucleic acid duplex provide templates for nucleic acid amplification. The platform technology for HDA is described in U.S. Pat. No. 7,282,328, “Helicase dependent amplification of nucleic acids,” which is incorporated by reference herein in its entirety.

Unlike other approaches for in vitro amplification of nucleic acids that use heat to separate nucleic acid duplexes, HDA uses one or more helicases. The separated nucleic acid strands serve as single-stranded templates for in vitro amplification of nucleic acids. Sequence-specific primers hybridize to the templates and are then extended by DNA polymerases to amplify an HDA target nucleic acid. This process repeats itself so that exponential amplification can be achieved at a single temperature.

A diversity of helicases can be used for HDA. Non-limiting examples of helicases for HDA are described in U.S. Pat. No. 7,282,328 and U.S. Pat. No. 7,662,594. For example, a thermostable helicase, which is a helicase that is capable of unwinding double-stranded DNA under elevated temperatures (e.g., preferred reaction temperature above about 60° C.), can be used in HDA. Examples of thermostable helicases include UvrD-like helicases. For example, Tte UvrD helicase is a helicase from the thermophilic organism Thermoanaerobacter tengcongensis.

In various aspects of the present disclosure, a helicase selected for use in HDA methodology is a thermostable helicase. In some embodiments, the helicase is a Tte-UvrD helicase.

Regions of nucleic acid strands that have been separated by one or more helicases can be amplified as part of HDA methodology. One or more polymerases are used for amplification. If the nucleic acid to be amplified is DNA, a DNA polymerase can be used for amplification. When the initial nucleic acid provided for an HDA method is RNA, a reverse transcriptase is used to first copy the RNA (or a region of the RNA) into a cDNA molecule and the cDNA is amplified by a DNA polymerase.

The DNA polymerase acts on the HDA target nucleic acid to extend the primers hybridized to the nucleic acid templates in the presence of dNTPs to form primer extension products complementary to the nucleotide sequence on the nucleic acid template.

DNA polymerases for HDA may be selected from polymerases lacking 5′ to 3′ exonuclease activity and which additionally may optionally lack 3′-5′ exonuclease activity. In some embodiments, the polymerase used according to the present disclosure is a thermostable polymerase. In some embodiments, the polymerase is Gst Polymerase. In some embodiments, the polymerase is WarmStart Gst polymerase, which is a Gst polymerase that has been modified to function at about 45° C. or higher. In some embodiments, the polymerase is Bst polymerase. In some embodiments, the polymerase is WarmStart Bst polymerase, which is a Bst polymerase that has been modified to function at about 45° C. or higher.

In some embodiments, HDA methods of the disclosure are also referred to as “thermostable helicase-dependent amplification” (tHDA). As used herein, tHDA is a type of HDA that uses a thermostable helicase and a thermostable polymerase. The thermostable properties of the helicase and polymerase enable performing HDA at high temperatures (e.g., 45° C.-75° C.), which may increase the specificity of target nucleic acid amplification.

Generally, primers suitable for use in HDA are short synthetic oligonucleotides, for example, having a length of more than 10 nucleotides and less than 50 nucleotides. Oligonucleotide primer design involves various parameters such as string-based alignment scores, melting temperature, primer length and GC content (Kampke et al., Bioinformatics 17:214-225 (2003)). When designing a primer, one of the important factors is to choose a sequence within the target fragment which is specific to the nucleic acid molecule to be amplified. Another important factor is to decide the melting temperature of a primer for HDA reaction. The melting temperature of a primer is determined by the length and GC content of that oligonucleotide. Preferably, the melting temperature of a primer is about equal to 10° C. higher than the temperature at which the hybridization and amplification will take place. For example, if the temperature of the hybridization and amplification is 60° C., the melting temperature of a pair of primers designed for that reaction should be in a range between 65° C. and 75° C. In preferred embodiments, the melting temperature of primers according to the present disclosure is about 65° C.

Each primer hybridizes to each end of the HDA target nucleic acid and may be extended in a 5′ to 3′ direction by a polymerase using the target nucleotide sequence (or complementary sequence) as a template. To achieve specific amplification, a homologous or perfect match primer is preferred. However, primers may include sequences at the 5′ end which are non-complementary to the target nucleotide sequence(s). Alternatively, primers may contain nucleotides or sequences throughout that are not exactly complementary to the HDA target nucleic acid. Primers may represent analogous primers or may be non-specific or universal primers for use in HDA as long as specific hybridization can be achieved by the primer-template binding at a predetermined temperature.

HDA methods may include more than one pair of primers. HDA methods using more than one pair of primers may be used to amplify nucleic acids comprising different target sequences of HDA.

In addition to helicase(s), polymerases, and primers, HDA methods may also use single stranded binding proteins (SSB). Some helicases show improved activity in the presence of SSB, which can stabilize unwound single-stranded nucleic acids so that they do not re-anneal. In some embodiments where a thermostable helicase is used, the presence of a single stranded binding protein is optional.

HDA methods may also use one or more accessory proteins. The term “accessory protein” refers to any protein capable of stimulating helicase activity. For example, E. coli MutL protein is an accessory protein for enhancing UvrD helicase melting activity. In some embodiments, accessory proteins are desirable for use with selected helicases. In alternative embodiments, unwinding of nucleic acids may be achieved by helicases in the absence of accessory proteins.

Other components that may be used in HDA include one or more buffers, one or more chemical reagents, one or more small molecules, salts (e.g., MgSO₄, KCl and NaCl), additives, and/or excipients. Examples of additives include Dithiothreitol (DTT) and Tween-20. Examples of excipients include sucrose, dextran and BSA.

In some embodiments, components for HDA comprise one or more buffers, salts (e.g., MgSO₄, KCl and NaCl), additives, and/or excipients. Examples of additives include Dithiothreitol (DTT) and Tween-20. Examples of excipients include sucrose, dextran and BSA. Sucrose, dextran and BSA are inert components for lyophilization.

In various embodiments of the present disclosure, HDA occurs in the presence of a set of components comprising a helicase, an energy source, DNA polymerase, deoxynucleotide triphosphate (dNTPs) and primers. Examples of an energy source are nucleotide triphosphates (NTPs) or dNTPs. In some embodiments, the set of components further comprise a single stranded binding protein. An example of a single stranded binding protein is the thermophilic archaeal Sulfolobus solfataricus SSB (SSo-SSB). In some embodiments, the set of components further comprise a Tris buffer, MgSO₄, KCl, NaCl, DTT, Tween-20, sucrose, dextran and BSA.

In various embodiments of the present disclosure, reverse transcription of RNA is combined with amplification of the resulting cDNA via HDA. For example, an initial target RNA in a provided sample is reverse transcribed to form target cDNA, and the target cDNA is amplified by HDA. An illustrative non-limiting example of a method that combines reverse transcription of RNA with amplification of the resulting cDNA via HDA is shown in FIG. 17 of U.S. Pat. No. 7,662,594. Methods of the present disclosure that combine reverse transcription and HDA include a reaction or a series of reactions that comprise reverse transcription, helicase-dependent denaturation and amplification. Either a reverse transcriptase or a polymerase with reverse transcription properties can be used to synthesize cDNA by reverse transcription of target RNA. Examples of reverse transcriptases include mutants of or wild-type Moloney Murine Leukemia Virus (MMLV) reverse transcriptase and mutants of or wild-type Avian Myeloblastosis Virus (AMV) reverse transcriptase. In some embodiments, a reverse transcriptase used according to the present disclosure is a mutant of the MMLV reverse transcriptase referred to as NxtScript Reverse Transcriptase (Roche Custom Biotech).

In some embodiments, methods for detecting target bacteria provided herein combine reverse transcription of a target RNA with amplification of the resulting cDNA. In various embodiments, reverse transcription and DNA amplification occur at the same time and in the same reaction vessel. A first strand cDNA is synthesized by reverse transcription of the target RNA, forming a DNA/RNA duplex. A helicase unwinds the DNA/RNA duplex into at least partial single strand nucleic acids and a SSB stabilizes the single strand nucleic acids. The single-stranded RNA enters a next round of reverse transcription (RT) reaction, generating more first strand cDNA. The single-stranded DNA is converted into double-stranded DNA by DNA polymerase and amplified concurrently in the HDA reaction. This process repeats itself to achieve exponential amplification of the RNA target sequence.

In various embodiments comprising reverse transcription of RNA combined with amplification of the resulting cDNA via HDA, a pair of sequence-specific primers is used for the reverse transcription and amplification reactions. In some embodiments, a pair of sequence-specific primers, one hybridizing to the 3′ end of the target nucleic acid (e.g., the target RNA, such as the 23S rRNA) and the other hybridizing to the 3′ end of the complimentary strand which is produced by reverse transcription (i.e., the target cDNA), are used. Subsequently, the pair of primers hybridize to the analogous strands in the amplified products.

In some embodiments, a pair of primers selected for HDA methods of the present disclosure are the primer pairs described above and in Example 1. In some embodiments, a pair of primers selected for HDA methods comprise nucleic acid sequences of SEQ ID NO: 2 and SEQ ID NO: 3. In some embodiments, a pair of primers selected for HDA methods consist of nucleic acid sequences of SEQ ID NO: 2 and SEQ ID NO: 3.

In some embodiments, reverse transcription and HDA reactions occur in a single reaction vessel with a single buffer, such that cDNA copies of the RNA target sequence act as a template for DNA amplification at the same time as more cDNA is generated from RNA by reverse transcription. Helicases that unwind both RNA-DNA duplexes and DNA duplexes are preferred in reactions that occur in a single reaction vessel. Such a helicase can be, for example, Tte-UvrD helicase. In some embodiments, reverse transcription and amplification are performed isothermally. An advantage of these embodiments is that unwinding by helicase and amplification can effectively occur at a single temperature. Methods of the present disclosure that combine reverse transcription and HDA can be used to detect and/or quantify target bacteria using HDA methodology.

In some embodiments, reverse transcription and HDA occur in the presence of a set of components comprising a helicase, an energy source, DNA polymerase, reverse transcriptase, dNTPs and primers. In some embodiments, the set of components further comprise a single stranded binding protein. In some embodiments, the set of components further comprise a Tris buffer, MgSO₄, KCl, NaCl, DTT, Tween-20, sucrose, dextran and BSA.

### Amplicon Contamination Control for Helicase-Dependent Amplification (HDA)

In some embodiments of the present disclosure, HDA compositions (e.g., mixtures comprising HDA components) and/or HDA methods are modified to incorporate components and/or methods for amplicon control. As used herein, the term “amplicon control” refers to reducing or eliminating carryover contamination. Amplicon control helps prevent prior positive reactions from contaminating and triggering false positives on subsequent negative reactions.

In some embodiments, amplicon control involves a reaction that occurs before reverse transcription and amplification reactions described herein. Such an amplicon control reaction destroys or removes contamination amplicon. In some embodiments, an amplicon control reaction involves at least two enzymes that enable destruction of contamination amplicon.

In some embodiments, an amplicon control reaction occurs in the same tube as HDA. In some embodiments, an amplicon control reaction must not occur during DNA amplification as it would destroy any amplified target (amplicon).

In some embodiments, an amplicon control reaction occurs at a different temperature as HDA. In some embodiments, an amplicon control reaction requires a temperature of about 37° C. In some embodiments, one or more enzymes involved in amplicon control have to be inactivated at the reverse transcription and amplification step of HDA (about 65° C.). In some embodiments, at least one enzyme involved in amplicon control is inactivated at temperatures higher than about 50° C.

In some embodiments, components for amplicon control comprise: (i) an enzyme that binds uracil in a DNA strand and converts it into an apurinic site; (ii) an enzyme that cleaves DNA at apurinic sites; and (iii) a specialized dNTP that is recognized by the enzyme of (i).

In some embodiments, components for amplicon control comprise: (i) uracil DNA glycosylase (also referred to as “UDG” or “UNG”); (ii) Endonuclease VIII; and (iii) dUTP. In some embodiments, the UDG is a thermolabile UDG. A non-limiting example of a UDG that may be used as described herein is Antarctic Thermolabile UDG enzyme (New England Biolabs, Ipswich, Mass.).

In some embodiments, dUTP is incorporated into all amplicons in an HDA method by Gst polymerase. As disclosed herein, the inclusion of dUTP does not inhibit Gst polymerase or the reverse transcriptase. The concentration of dUTP should be optimized to sufficiently remove unwanted amplicon without slowing the reaction significantly.

### Detection and/or Quantification of Target Nucleic Acids

Various methods and instruments can be used to detect and/or quantify target nucleic acids in conjunction with other methods of the present disclosure. As used herein, “to detect” means to identify the presence or absence of the target nucleic acids and “to quantify” means to measure or calculate the quantity of the target nucleic acids. In some embodiments, detection and quantification occur concomitantly. In some embodiments, detection and quantification are achieved by the same means.

In various embodiments, detection and/or quantification comprises identifying and/or measuring amplified nucleic acids, for example, the amplicon products of HDA.

Amplified nucleic acid products may be identified and/or measured by methods including ethidium-bromide staining or by means of a label selected from the group consisting of a radiolabel, a fluorescent label, and an enzyme.

Fluorescence measurement is a type of detection/quantification method that can be used for detection and/or quantification of amplified nucleic acids of the present disclosure. For example a fluorescent intercalator that is only fluorescent when bound to dsDNA, may be used for fluorescence measurement. Alternatively, fluorescent probes may be designed to only fluoresce when bound to specific nucleic acid sequences, rather than any dsDNA. In some embodiments, amplified target nucleic acids can be detected/quantified using quenched fluorescent oligonucleotides that generate fluorescence when bound to or incorporated into an amplification product. Fluorescence can be measured using an instrument called a fluorometer.

Real-time measurement of nucleic acid amplification is also a type of detection/quantification method. In real-time measurement, the progress of nucleic acid amplification is monitored as it occurs (i.e., in real time). Measurements are therefore collected throughout the process of amplification, rather than at the end of amplification. Many real-time methods use fluorescence as the readout for nucleic acid amplification. Real-time reactions are characterized by the point in time during cycling when amplification of a target is first detected rather than the amount of target accumulated after a fixed number of amplification cycles. The higher the starting copy number of the nucleic acid target, the sooner a significant increase in signal (e.g, fluorescence) is observed. In contrast, an endpoint assay measures the amount of accumulated product at the end of the amplification. In various methods of the present disclosure, real-time measurement of nucleic acid amplification is used for detection and/or quantification of a target nucleic acid. In some embodiments, real-time measurement is used to detect the amplicon products of HDA methods of the disclosure. In some embodiments, the step of detecting according to a method of the present disclosure comprises real-time measurement of amplified target cDNA.

In various embodiments, detection and/or quantification comprises identifying and/or measuring the amplicon products of HDA. In some embodiments, detection and/or quantification of the amplicon products of HDA comprises subjecting the assay mixture to successive cycles of amplification to generate a signal from a probe designed to detect the amplicon (for example, a fluorescent signal) and quantifying the nucleic acid presence. In some embodiments, quantifying the nucleic acid presence is based on the signal cycle threshold of the amplification reaction. In some embodiments, the step of detecting according to a method of the present disclosure comprises quantifying the presence of amplified target cDNA based on a fluorescence signal cycle threshold of the amplification reaction. In some embodiments, quantifying the nucleic acid presence is based on the strength of a detection signal.

In some embodiments, the step of detecting presence or absence of amplified target cDNA according to a method of the present disclosure comprises determining fluorescence signal from a fluorescent molecular probe as an indication of presence based on the speed of the signal. In some embodiments, the step of detecting further comprises determining fluorescence signal from the probe as an indication of presence based on the rate at which signal increases and the strength of the signal. In some embodiments, the step of detecting presence or absence of the amplified target cDNA comprises: (i) detecting the speed of the signal and the strength of the signal that meets a threshold speed and strength and thereby determining the presence of the target bacteria in a sample (e.g., environmental sample); or (ii) detecting that the speed of the signal and the strength of the signal does not meet a threshold speed and strength and thereby determining the absence of the target bacteria in the sample.

In some embodiments, an algorithm is used for detection and/or quantification. As a non-limiting example for a molecular probe (e.g., a fluorescent molecular probe), an algorithm can be used to determine signal from the probe as an indication of presence based on the speed of the signal, as determined by the Cq (when fluorescence intensity exceeds a noise threshold) and the slope (the rate at which fluorescence increases), and the strength of the signal (maximum fluorescence). If the generated signal meets the threshold speed and strength criteria, it will be called “present” (positive signal). If it does not meet these criteria, it will be called as “absent” (negative signal). In some embodiments, the speed of the signal and the strength of the signal is considered to meet the threshold speed and strength criteria if it matches or surpasses the threshold values set for speed and strength. In some embodiments, a speed signal is considered to match or surpass the threshold value set for speed if it exceeds a noise threshold at or before the speed threshold value. In some embodiments, a strength signal is considered to match or surpass the threshold value set for strength if the maximum fluorescence matches or surpasses the strength threshold value.

In some embodiments, the presence and absence assay signal are reliant on the limit of detection of the assay. Samples with a target concentration (for example, the concentration of Salmonella rRNA prior to reverse transcription and amplification) greater than the limit of detection will have a strong fluorescent signal with a small Cq (fast onset) and will be called as present. However, for samples with target concentrations close to the limit of detection, an absence signal does not mean an absolute absence of the target. Usually multiple replicates are necessary to detect presence at these levels. For samples with a much lower target concentration than the limit of detection of the assay, the amount of target present in the sample will not be enough to generate a fast and a strong fluorescent signal and will fail to meet the criteria for making a positive call, therefore, will be called as absent/negative.

Amplification assay results may be read by an automated reader, such as a reader that measure fluorescence. Alternatively or additionally, assay results may be detected by enzymatic detection methods or gel electrophoresis.

In some embodiments, detection of amplified nucleic acids comprises quantification of the amplified product (e.g., quantification of the amplicon). In some embodiments, quantification comprises measuring relative levels of a readout, such as relative levels of a test readout (e.g., a readout indicating presence of a target nucleic acid) relative to a control readout. In some embodiments, the readout is a fluorescence readout.

In some embodiments, detection of amplified nucleic acids comprises detecting presence or absence of the amplified product.

In some embodiments, the amplicon comprises the nucleic acid sequence (read in the 5′ to 3′ direction) of GAG AAG GCA CGC TGA CAC GTA GGT GAA GTG ATT TAC TCA CGG AGC TGA AGT CAG (SEQ ID NO: 4). In some embodiments, the amplicon comprises a nucleic acid sequence having at least 90%, 91%, 92%, 93%, 94%, 95%, 96%, 97%, 98%, or 99% but less than 100% sequence identity to SEQ ID NO: 4. In some embodiments, the amplicon consists of the nucleic acid sequence of SEQ ID NO: 4.

In some embodiments, one or more target nucleic acids are amplified according to HDA methods and the amplified nucleic acid is detected using a probe designed to identify the amplified nucleic acid. In some embodiments, an initial target RNA present in a provided sample is reverse transcribed to form target cDNA, the target cDNA is amplified by HDA, and the amplified target cDNA is detected.

In some embodiments, the probe used to detect amplified nucleic acids of the present disclosure is a fluorescent hybridization probe. In some embodiments, the fluorescent hybridization probe is a conditional fluorescent hybridization probe that emits fluorescence when hybridized to a nucleic acid molecule. In some embodiments, a conditional fluorescent hybridization probe comprises a fluorophore and a quencher that prevents the fluorophore from generating fluorescence unless the probe is bound to the amplified nucleic acid being detected.

In some embodiments, the conditional fluorescent hybridization probe comprises the nucleic acid sequence of: CCA TGA GTA AAT rCAC TTC ACC TAC GTG (SEQ ID NO: 5), wherein the “r” denotes that the base is RNA. In some embodiments, the conditional fluorescent hybridization probe comprises a nucleic acid sequence with at least 90% sequence identity to SEQ ID NO: 5. In some embodiments, a fluorophore may be linked at either the 5′ or 3′ end of the probe. In some embodiments, a quencher may be linked at the opposite end of the probe to that of the fluorophore.

In some embodiments, the conditional fluorescent hybridization probe comprises 5′/56-ROXN/CCA TGA GTA AAT rCAC TTC ACC TAC GTG/3IAbRQSp/3′ (also referred to by the present disclosure as the “Sal ROX probe”), wherein “56-ROXN” is ROX (carboxy-X-rhodamine) fluorophore; “3IAbRQSp” is Iowa Black RQ-Sp Quencher; and the “r” denotes that the base is RNA. This probe comprises DNA bases and a single RNA base. When the probe binds the nucleic acid being detected, the RNA base can be recognized by an enzyme (e.g., RNase H2) that cleaves the probe, thereby separating the quencher and the fluorophore and generating fluorescence.

In some embodiments, detection of amplified nucleic acid occurs in the same reaction vessel as nucleic acid amplification. In some embodiments, detection of amplified nucleic acid occurs in the same reaction vessel as reverse transcription of an initial target RNA to form target cDNA and amplification by HDA of the target cDNA; in this scenario, the nucleic acid being detected is the amplified cDNA. In some embodiments, the amplified cDNA is detected/quantified using an instrument that supports isothermal DNA/RNA amplification methods. In some embodiments, the instrument takes fluorescence measurements in real-time. As non-limiting examples, the instrument may be a Genie® II or Genie® III reader (made by OptiGene, UK). In some embodiments, the instrument used according to methods of the present disclosure is a Genie® II reader (OptiGene, UK).

In some embodiments, detection of amplified nucleic acid comprises use of a detection mixture comprising a set of detection mixture components. In some embodiments, the set of detection mixture components comprises a probe. In some embodiments, the probe is a conditional fluorescent hybridization probe. In some embodiments, the conditional fluorescent hybridization probe emits fluorescence when hybridized to a nucleic acid molecule comprising the nucleic acid sequence of: CAC GTA GGT GAA GTG ATT TAC TCA CGG (SEQ ID NO: 6), or a sequence with at least 90% sequence identity to SEQ ID NO: 6. In some embodiments, the conditional fluorescent hybridization probe emits fluorescence when hybridized to a nucleic acid molecule comprising a nucleic acid sequence having at least 90%, 91%, 92%, 93%, 94%, 95%, 96%, 97%, 98%, or 99% sequence identity to SEQ ID NO: 6. In some embodiments, the conditional fluorescent hybridization probe emits fluorescence when hybridized to a nucleic acid molecule comprising a nucleic acid sequence having 100% sequence identity to SEQ ID NO: 6. In some embodiments, the conditional fluorescent hybridization probe emits fluorescence when hybridized to a nucleic acid molecule comprising the nucleic acid sequence of: CAC GTA GGT GAA GTG ATT TAC TCA TGG (SEQ ID NO: 7), or a sequence with at least 90% sequence identity to SEQ ID NO: 7. In some embodiments, the conditional fluorescent hybridization probe emits fluorescence when hybridized to a nucleic acid molecule comprising a nucleic acid sequence having at least 90%, 91%, 92%, 93%, 94%, 95%, 96%, 97%, 98%, or 99% sequence identity to SEQ ID NO: 7. In some embodiments, the conditional fluorescent hybridization probe emits fluorescence when hybridized to a nucleic acid molecule comprising a nucleic acid sequence having 100% sequence identity to SEQ ID NO: 7.

In some embodiments, the set of detection mixture components comprises a Sal ROX probe and RNase H2.

In some embodiments, the set of detection mixture components further comprises any of the HDA components described above.

In some embodiments, the set of detection mixture components comprises components for HDA and reverse transcription.

In some embodiments, the set of detection mixture components further comprises components for amplicon control, such as the components described above.

In some embodiments, the set of detection mixture components comprises one or more of: a Sal ROX probe, RNase H2, a helicase, an energy source, DNA polymerase, reverse transcriptase, dNTPs and primers. In some embodiments, the set of detection mixture components comprises a Sal ROX probe, RNase H2, a helicase, an energy source, DNA polymerase, reverse transcriptase, dNTPs and primers. In some embodiments, the set of components further comprises a single stranded binding protein. In some embodiments, the set of components further comprises a UDG, an enzyme that cleaves DNA at apurinic sites, and dUTP. In some embodiments, the set of components further comprises a Tris buffer, MgSO₄, KCl, NaCl, DTT, Tween-20, sucrose, dextran and/or BSA.

In some embodiments, the primers of a detection mixture comprise a first primer having hybridization specificity for a single-stranded nucleic acid region comprising a nucleic acid sequence of the target RNA and a second primer having hybridization specificity for a single-stranded nucleic acid region comprising a nucleic acid sequence complementary to the target RNA sequence.

In some embodiments, the set of detection mixture components further comprises a control RNA and a control probe that is able to detect the control RNA. In some embodiments, the control probe is able to detect amplification products of the control RNA. In some embodiments, amplification products of the control RNA are amplified by the same primers as those used for reverse transcription and HDA in the detection mixture.

In some embodiments, the detection mixture is lyophilized.

In some embodiments, a detection mixture contacted with a composition or mixture comprising a nucleic acid template for the nucleic acid to be detected by components in the detection mixture generates an assay mixture. As used herein, an “assay mixture” refers to a mixture comprising components of a detection mixture and a nucleic acid template for amplification of nucleic acids detected by the probe in the detection mixture. In some embodiments, the nucleic acid template is an RNA (or a region of the RNA) that is reverse transcribed in the assay mixture into a cDNA and the cDNA is amplified and detected by the probe. In some embodiments, an assay mixture is generated when a lysate or an aliquot of a lysate is contacted with a detection mixture. In some embodiments, a method of the present disclosure comprises reverse transcribing a target RNA of a target bacteria in the assay mixture to form target cDNA and amplifying the target cDNA by helicase-dependent amplification (HDA). In some embodiments, a method of the present disclosure also comprises detecting presence or absence of the amplified target cDNA, thereby determining the presence or absence of the target bacteria in the sample.

### Salmonella Detection Assays

The present disclosure provides methods for detection of Salmonella spp. from environmental samples. In various embodiments, the present disclosure provides methods of detecting Salmonella from an environmental sample without the need for enrichment or a prolonged incubation period. The provided methods for Salmonella detection combine various individual methods described above (e.g., pre-treatment, lysis, HDA, amplicon control and/or detection/quantification of target nucleic acids) for the purposes of detecting and/or quantifying Salmonella from a sample.

In some embodiments, methods for detecting Salmonella provided herein combine reverse transcription of a target RNA with amplification of the resulting cDNA. In various embodiments, reverse transcription and DNA amplification occur at the same time and in the same reaction vessel. Descriptions of concurrent reverse transcription and DNA amplification are provided above, for example, in the “Helicase-dependent amplification” section.

In addition to the above-described concurrent reverse transcription and DNA amplification, the provided methods for detection of Salmonella also combine various other methods described throughout the present disclosure to detect and/or quantify Salmonella in a sample.

A non-limiting example of the combination of methods from the present disclosure that can be used for detection of Salmonella comprise: (i) combined reverse transcription and DNA amplification via thermostable helicase-dependent amplification (tHDA) and (ii) detection of amplified nucleic acids.

Another non-limiting example of the combination of methods from the present disclosure that can be used for detection of Salmonella comprise: (i) bacterial cell lysis; (ii) combined reverse transcription and DNA amplification via thermostable helicase-dependent amplification (tHDA); and (iii) detection of amplified nucleic acids.

Another non-limiting example of the combination of methods from the present disclosure that can be used for detection of Salmonella comprise: (i) sample pre-treatment to remove nucleic acids not associated with intact cells; (ii) bacterial cell lysis; (iii) combined reverse transcription and DNA amplification via thermostable helicase-dependent amplification (tHDA); and (iv) detection of amplified nucleic acids.

Another non-limiting example of the combination of methods from the present disclosure that can be used for detection of Salmonella comprise: (i) sample pre-treatment to remove nucleic acids not associated with intact cells; (ii) bacterial cell lysis; (iii) amplicon control to prevent prior positive reactions from contaminating and triggering false positives on subsequent negative reactions; (iv) combined reverse transcription and DNA amplification via thermostable helicase-dependent amplification (tHDA); and (v) detection of amplified nucleic acids.

In various embodiments, detection of amplified nucleic acids comprises use of at least one fluorescent hybridization probe that recognizes the amplified nucleic acids.

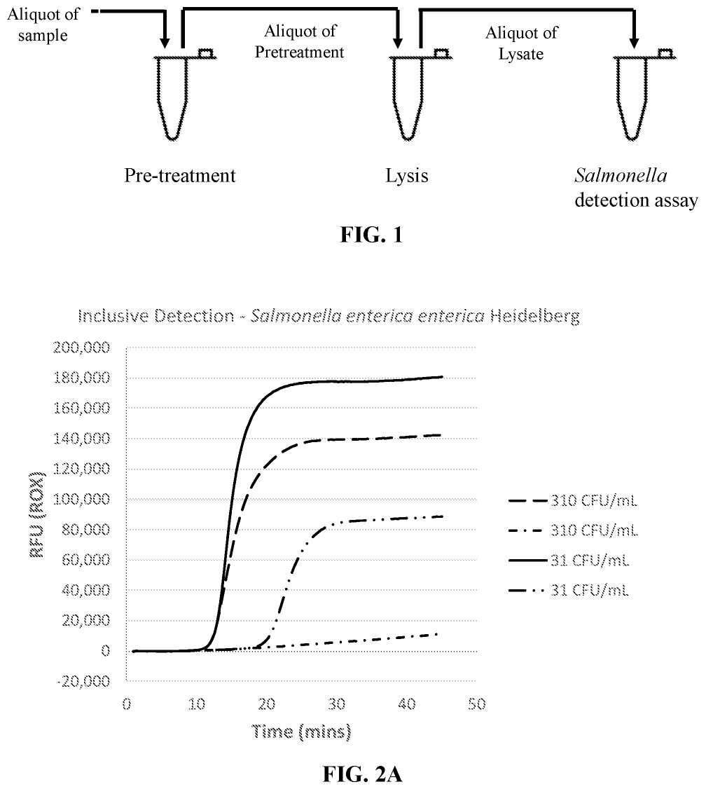

In various embodiments of the present disclosure, the combination of methods for detection of Salmonella in a provided sample are combined in a series of steps that occur in the following sequence: (i) sample pre-treatment to remove nucleic acids not associated with intact cells; (ii) cell lysis; (iii) amplicon control; and (iv) reverse transcription, DNA amplification, and detection of amplified target. An illustrative sequence of steps for a Salmonella detection method is provided in FIG. 1. In various embodiments, nucleic acid amplification occurs at a single temperature (usually between about 55-68° C.). In some embodiments, nucleic acid amplification occurs at or above 45° C. and at or below 70° C.

In some embodiments, the combination of methods for detection of Salmonella comprises use of a pre-treatment mixture, a lysis mixture, and/or a detection mixture. For example, any of the above-described pre-treatment, lysis and detection mixtures may be used for detection of Salmonella according to the present disclosure.

As a non-limiting example, the pre-treatment mixture comprises: (i) micrococcal nuclease, (ii) CaCl₂; (iii) Tris-HCl, pH 8.8 (iv) BSA; (v) dextran; and (vi) sucrose. In some embodiments, the pre-treatment mixture is lyophilized. In some embodiments, the set of pre-treatment mixture components comprises the components listed in Table 2 and the concentration of each of the listed components after resuspension of the lyophilized pre-treatment mixture is the concentration listed in Table 2.

As a non-limiting example, the lysis mixture comprises: (i) lyosozyme; (ii) mutanolysin; (iii) proteinase K; (iv) achromopeptidase; (v) Chelex®-100; (vi) Tris HCl, pH 8.8; (vii) EGTA; (viii) dextran; and (ix) sucrose. In some embodiments, the lysis mixture components are lyophilized as one or more lyophilized pellets. In some embodiments, the set of lysis mixture components comprises the components listed in Table 4 and the concentration of each of the listed components after resuspension of the lyophilized lysis mixture components is the concentration listed in Table 4.

As a non-limiting example, the detection mixture comprises: (i) a Sal ROX probe, (ii) RNase H2, (iii) a helicase, (iv) an energy source in the form of dATP, (v) DNA polymerase, (vi) reverse transcriptase, (vii) dNTPs, (viii) forward and reverse primers, (ix) Tris-HCl, pH 8.8, (x) KCl, (xi) NaCl, and (xii) magnesium sulfate. In some embodiments, the detection mixture further comprises at least one single stranded binding protein (SSB). In some embodiments, the detection mixture further comprises: (i) UDG, (ii) Endonuclease VIII, and (iii) dUTP. In some embodiments, the detection mixture further comprises: (i) DTT, (ii) Tween-20, (iii) sucrose, (iv) dextran, and/or (v) BSA. In some embodiments, the detection mixture further comprises a control RNA and a control probe that is able to detect the control RNA. In some embodiments, the detection mixture comprises one or more of the components listed in Table 5. In some embodiments, one or more components of the detection mixture are lyophilized together as one lyophilized pellet. In some embodiments, one or more components of the detection mixture are lyophilized separately as more than one lyophilized pellet.

In some embodiments, the forward and revers primers of a detection mixture comprise a first primer having hybridization specificity for a single-stranded nucleic acid region comprising a nucleic acid sequence of the target RNA and a second primer having hybridization specificity for a single-stranded nucleic acid region comprising a nucleic acid sequence complementary to the target RNA sequence.

In some embodiments, the detection mixture is lyophilized. In some embodiments, one or more lyophilized pellets, each comprising one or more components of a detection mixture are resuspended. For example, the one or more lyophilized pellets may be resuspended with a liquid composition. In some embodiments, the one or more lyophilized pellets may be resuspended with a sample or solution comprising lysed target bacteria cells (e.g., a lysate or an aliquot thereof). In some embodiments, the concentration of one or more of the components of the detection mixture after resuspension is in the range of concentrations listed in Table 5. In some embodiments, the set of detection mixture components comprises the components listed in Table 5 and the concentration of each of the listed components after resuspension of the lyophilized detection mixture components is in the range specified in Table 5. In some embodiments, the set of detection mixture components comprises one or more of the components listed in Table 6 and the concentration of each component after resuspension of the lyophilized detection mixture components is the concentration listed in Table 6. In some embodiments, the set of detection mixture components comprises the components listed in Table 6 and the concentration of each of the listed components after resuspension of the lyophilized detection mixture components is the concentration listed in Table 6.

TABLE 5
Concentration ranges for detection mixture components

| Component | Concentration range | |
|---|---|---|
| Tris HCl pH 8.8 | 15-65 | mM |
| KCl | 8-12 | mM |
| NaCl | 22-50 | mM |
| dGTP | 0.4-1 | mM |
| dCTP | 0.4-1 | mM |
| dTTP | 0-1 | mM |
| dATP | 5.5-7.5 | mM |
| dUTP | 0-1 | mM |
| Forward Primer | 60-100 | nM |
| Reverse Primer | 60-100 | nM |
| Probe | 40-100 | nM |
| Control HDA Probe | 0-100 | nM |
| Warmstart Gst DNA Polymerase | 1.8-5 | Units/μL |
| Tte-UvrD Helicase | 10-20 | ng/μL |
| Sso-SSB | 2-20 | ng/μL |
| Nexscript Reverse Transcriptase | 0.3-0.8 | Units/μL |
| RNaseH2 | 1-20 | ng/μL |
| Antarctic Thermolabile UDG | 0-0.2 | Units/μL |
| Endonuclease VIII | 0-0.6 | Units/μL |
| Control RNA | 0-500,000 | Copies/μL |
| DTT | 1.5-3 | mM |
| Tween-20 | 0-0.2% | (v/v) |
| Sucrose | 1-6% | (m/v) |
| Dextran | 1-5% | (m/v) |
| MgSO4 | 8-12 | mM |
| BSA | 0-1 | mg/mL |

TABLE 6
Detection mixture components and concentrations

| Component | Concentration | |
|---|---|---|
| Tris HCl pH 8.8 | 20.4 | mM |
| KCl | 10 | mM |
| NaCl | 24.8 | mM |
| dGTP | 0.5 | mM |
| dCTP | 0.5 | mM |
| dTTP | 0.2 | mM |
| dATP | 6.6 | mM |
| dUTP | 0.3 | mM |
| Forward Primer | 80.7 | nM |
| Reverse Primer | 80.7 | nM |
| Probe | 80.7 | nM |
| Control HDA Probe | 80.7 | nM |
| Warmstart Gst DNA Polymerase | 2 | Units/μL |
| Tte-UvrD Helicase | 11.5 | ng/μL |
| Sso-SSB | 2 | ng/μL |
| Nexscript Reverse Transcriptase | 0.4 | Units/μL |
| RNaseH2 | 5.8 | ng/μL |
| Antarctic Thermolabile UDG | 0.02 | Units/μL |
| Endonuclease VIII | 0.4 | Units/μL |
| Control RNA | 99600 | Copies/μL |
| DTT | 2 | mM |
| Tween-20 | 0.14% | (v/v) |
| Sucrose | 2% | (m/v) |
| Dextran | 2.5% | (m/v) |
| MgSO4 | 10 | mM |
| BSA | 0.5 | mg/mL |

In some embodiments, the forward primer of the detection mixture comprises the nucleic acid sequence of 5′ GAG AAG GCA CGC TGA CAC 3′(SEQ ID NO: 2) and the reverse primer comprises the nucleic acid sequence of 5′ CTG ACT TCA GCT CCG TGA GTA AAT 3′ (SEQ ID NO: 3).

In some embodiments, methods for detecting Salmonella provided herein comprise determining the presence or absence of Salmonella in an environmental sample. In some embodiments, the environmental sample is from an environment comprising a low concentration of Salmonella and the methods described herein are of sufficient sensitivity to detect the presence of Salmonella in the environmental sample. In some embodiments, methods described herein are of sufficient sensitivity to detect presence of Salmonella from a sample having as little as 30-60 CFU of Salmonella. In some embodiments, methods described herein are of sufficient sensitivity to detect presence of Salmonella from a sample having as little as 5-10 CFU of Salmonella. In some embodiments, the sample further comprises bacteria that is not Salmonella. In some embodiments, the environmental sample is from a solid surface comprising a low concentration of Salmonella and the methods described herein are of sufficient sensitivity to detect the presence of Salmonella in the environmental sample. In some embodiments, methods described herein are of sufficient sensitivity to detect presence of Salmonella in an environmental sample from a solid surface that comprises from about 5 to about 200 CFU of Salmonella per 1 square inch of the solid surface. In some embodiments, the solid surface comprises from about 5 to about 100 CFU of Salmonella per 1 square inch of the solid surface. In some embodiments, the solid surface comprises from about 5 to about 50 CFU of Salmonella per 1 square inch of the solid surface. In some embodiments, the solid surface further comprises microflora that is not Salmonella. In various embodiments wherein the sample or environment from where the sample is collected comprises Salmonella and microflora that is not Salmonella, the methods described herein are of sufficient sensitivity and specificity to detect presence of Salmonella.

### Compositions and Kits

The present disclosure also provides compositions and kits that may be used in the methods described herein.

In one aspect, the disclosure provides a lyophilized pre-treatment composition comprising one or more components of a pre-treatment mixture. In some embodiments, the lyophilized pre-treatment composition comprises (i) micrococcal nuclease, (ii) calcium chloride (CaCl₂); (iii) Tris buffer (e.g., Tris-HCl pH 8.8); and (iv) BSA. In some embodiments, the lyophilized pre-treatment composition further comprises sucrose. In some embodiments, the lyophilized pre-treatment composition further comprises dextran. In some embodiments, the concentration of each component of the lyophilized pre-treatment composition after resuspension (for example, resuspension with a liquid composition, e.g., resuspension with a sample or solution comprising target bacteria cells) is the concentration listed in Table 2.

In some embodiments, the present disclosure provides a lyophilized composition comprising micrococcal nuclease and calcium chloride (CaCl₂), wherein, upon resuspension of the lyophilized composition, the concentration of micrococcal nuclease ranges from 0.1-0.3 Units/μL, and the concentration of CaCl₂ ranges from 2-6 mM. In some embodiments, upon resuspension of the lyophilized composition, the concentration of micrococcal nuclease is 0.22 Units/μL, and the concentration of CaCl₂ is 4.1 mM.

In one aspect, the disclosure provides a lyophilized lysis composition comprising one or more components of a lysis mixture. In some embodiments, the lyophilized lysis composition comprises (i) at least one of lyosozyme and mutanolysin; (ii) at least one of proteinase K and achromopeptidase; and (iii) EGTA. In some embodiments, the concentration of each component of the lyophilized lysis composition after resuspension (for example, resuspension with a liquid composition, e.g., resuspension with a sample or solution comprising target bacteria cells or resuspension with a pre-treated sample or aliquot thereof) is the concentration listed in Table 4.

In some embodiments, the present disclosure provides a lyophilized composition comprising (i) at least one of lyosozyme and mutanolysin; (ii) at least one of proteinase K and achromopeptidase; and (iii) EGTA, wherein, upon resuspension of the lyophilized composition, the concentration of lysozyme ranges from 0-1 mg/mL, the concentration of mutanolysin ranges from 0-30 Units/mL, the concentration of proteinase K ranges from 0-1 mg/mL, the concentration of achromopeptidase ranges from 0-150 Units/mL, and the concentration of EGTA ranges from 2-5 mM. In some embodiments, upon resuspension of the lyophilized composition, the concentration of lysozyme is 0.8 mg/mL, the concentration of mutanolysin is 20 Units/mL, the concentration of proteinase K is 0.8 mg/mL, the concentration of achromopeptidase is 85.6 Units/mL, and the concentration of EGTA is 2.6 mM.

In one aspect, the disclosure provides a lyophilized detection mixture composition comprising one or more components of a detection mixture. The one or more components may be selected from components listed in Table 6.

In some embodiments, the disclosure provides a lyophilized composition comprising a first primer comprising the nucleic acid sequence of CTG ACT TCA GCT CCG TGA GTA AAT (SEQ ID NO: 3) or a sequence with at least 90% sequence identity to SEQ ID NO: 3 and a second primer comprising the nucleic acid sequence of GAG AAG GCA CGC TGA CAC (SEQ ID NO: 2) or a sequence with at least 90% sequence identity to SEQ ID NO: 2.

Aspects of the disclosure also include kits, the kits comprising components and/or compositions used in the methods described herein.

In some embodiments, a kit of the present disclosure may be for use in a method of determining the presence or absence of target bacteria in an environmental sample.

In some embodiments, a kit may comprise one or more of: a collection device, pre-treatment mixture or components thereof, lysis mixture or components thereof, detection mixture or components thereof, equipment (e.g., optical reader), reagents (primers, probes, dNTPs, enzymes, etc.), and instructions for use.

In some embodiments, a kit comprises a pre-treatment mixture, a lysis mixture, and a detection mixture. In some embodiments, one or more of the pre-treatment mixture, the lysis mixture, and the detection mixture are lyophilized. In some embodiments, one or more components of the pre-treatment mixture, the lysis mixture, and the detection mixture are lyophilized.

In one aspect, a kit comprises a first mixture and a second mixture, wherein the first mixture comprises micrococcal nuclease and a divalent salt and the second mixture comprises a divalent ion chelator, at least one lytic enzyme and at least one protease. For example, the divalent salt may be calcium chloride (CaCl₂) and the divalent ion chelator may be EGTA. In some embodiments, the at least one lytic enzyme comprises lyosozyme and mutanolysin and the at least one protease is proteinase K. In some embodiments, the kit further comprises a third mixture, the third mixture comprising a chelating resin such as Chelex-100. In some embodiments, the kit further comprises a fourth mixture, the fourth mixture comprising a helicase, an energy source for the helicase, a DNA polymerase, a reverse transcriptase, and dNTPs. In some embodiments, the fourth mixture further comprises a single stranded binding protein. In some embodiments, the fourth mixture further comprises (i) an enzyme that binds uracil in a DNA strand and converts it into an apurinic site; (ii) an enzyme that cleaves DNA at apurinic sites; and (iii) a specialized dNTP that is recognized by the enzyme of (i). In some embodiments, the fourth mixture further comprises a first primer having hybridization specificity for a single-stranded nucleic acid region comprising a nucleic acid sequence of the Salmonella 23S ribosomal RNA and a second primer having hybridization specificity for a single-stranded nucleic acid region comprising a nucleic acid sequence complementary to the nucleic acid sequence of the Salmonella 23S ribosomal RNA. For example, the first primer may comprise the nucleic acid sequence of 5′ CTG ACT TCA GCT CCG TGA GTA AAT 3′ (SEQ ID NO: 3) or a sequence with at least 90% sequence identity to SEQ ID NO: 3 and the second primer may comprise the nucleic acid sequence of 5′ GAG AAG GCA CGC TGA CAC 3′ (SEQ ID NO: 2) or a sequence with at least 90% sequence identity to SEQ ID NO: 2. In some embodiments, the fourth mixture further comprises at least one probe. The at least one probe may be a conditional fluorescent hybridization probe that emits fluorescence when hybridized to a nucleic acid molecule comprising the nucleic acid sequence of: (i) CAC GTA GGT GAA GTG ATT TAC TCA CGG (SEQ ID NO: 6), or a sequence with at least 90% sequence identity to SEQ ID NO: 6; or (ii) CAC GTA GGT GAA GTG ATT TAC TCA TGG (SEQ ID NO: 7), or a sequence with at least 90% sequence identity to SEQ ID NO: 7. In some embodiments, the at least one probe comprises the nucleic acid sequence of: CCA TGA GTA AAT rCAC TTC ACC TAC GTG (SEQ ID NO: 5), or a sequence with at least 90% sequence identity to SEQ ID NO: 5. In some embodiments, the first, second, third and/or fourth mixture is lyophilized. In some embodiments, the first mixture is lyophilized and upon resuspension of the first mixture, the concentration of micrococcal nuclease ranges from 0.1-0.3 Units/μL and the concentration of the divalent salt ranges from 2-6 mM. In some embodiments, the second mixture is lyophilized and upon resuspension of the second mixture, the concentration of the divalent ion chelator ranges from 2-5 mM.

In another aspect, a kit provided by the present disclosure comprises a first primer having hybridization specificity for a single-stranded nucleic acid region comprising a nucleic acid sequence of Salmonella 23S ribosomal RNA and a second primer having hybridization specificity for a single-stranded nucleic acid region comprising a nucleic acid sequence complementary to the nucleic acid sequence of the Salmonella 23S ribosomal RNA. In some embodiments, the first primer comprises the nucleic acid sequence of 5′ CTG ACT TCA GCT CCG TGA GTA AAT 3′ (SEQ ID NO: 3) or a sequence with at least 90% sequence identity to SEQ ID NO: 3 and the second primer comprises the nucleic acid sequence of 5′ GAG AAG GCA CGC TGA CAC 3′ (SEQ ID NO: 2) or a sequence with at least 90% sequence identity to SEQ ID NO: 2. In some embodiments, the kit further comprises at least one probe for detecting a nucleic acid molecule comprising the nucleic acid sequence of: (i) CAC GTA GGT GAA GTG ATT TAC TCA CGG (SEQ ID NO: 6), or a sequence with at least 90% sequence identity to SEQ ID NO: 6; or (ii) (ii) CAC GTA GGT GAA GTG ATT TAC TCA TGG (SEQ ID NO: 7), or a sequence with at least 90% sequence identity to SEQ ID NO: 7. In some embodiments, the kit comprises at least one probe that comprises the nucleic acid sequence of: CCA TGA GTA AAT rCAC TTC ACC TAC GTG (SEQ ID NO: 5), or a sequence with at least 90% sequence identity to SEQ ID NO: 5. In some embodiments, the first primer, the second primer and the probe are lyophilized.

### EXAMPLES

The following examples are offered by way of illustration, not by way of limitation.

### Example 1: Design of Primers and Probe for Amplification and Detection of Salmonella Target Nucleic Acids

The present example demonstrates methods for the design of primers and probes to amplify and detect nucleic acids from Salmonella species (spp.). The primers and probes designed according to the present example are specific to Salmonella enterica subspecies and serovars and are not specific to other bacteria such as Escherichia coli (E. coli).

Primers and Probe Design

RNA sequences for bacteria of interest (Salmonella spp.) and other bacteria (e.g., E. coli, C. freundii) were obtained from the National Center for Biotechnology Information (NCBI) GenBank and aligned using DNASTAR (Madison, Wis.) software. Forward and reverse primers and a probe were designed against Salmonella 23S rRNA sequence. The primers were designed to match tHDA primer design parameters and they were ordered from Integrated DNA Technologies (IDT) (Coralville, Iowa). Table 7 shows the sequences of the primers and probe.

TABLE 7
Example primer and probe sequences for
amplification and detection of
Salmonella 23S rRNA

| | | SEQ |
|---|---|---|
| | | ID |
| | Sequence | NO. |
| Forward primer | 5′ GAG AAG GCA CGC | 2 |
| | TGA CAC 3′ | |
| Reverse primer | 5′ CTG ACT TCA GCT | 3 |
| | CCG TGA GTA AAT 3′ | |
| Probe (the “r” | CCA TGA GTA AAT rCAC | 5 |
| denotes that | TTC ACC TAC GTG | |
| the base is RNA) | | |

Inclusivity, Exclusivity and LOD Testing

Overnight cultures were prepared for both inclusive and exclusive strains by growing them in Tryptic Soy Broth (TSB) at 37° C. The inclusive and exclusive strains tested in the present example are shown in Table 8 and Table 9. The cultures were serially diluted in Butterfield's Phosphate Buffer (BPB) to obtain the desired dilution. Then, 100 μL of desired dilutions were plated on Tryptic Soy Agar (TSA) plates and incubated overnight at 37° C. to obtain the titers for each overnight culture.

TABLE 8
Inclusive strains

| Strain | ATCC # |
|---|---|
| S. Heidelberg | 8326 |
| S. Oranienberg | 9239 |
| S. Muenchen | 8388 |
| S. Berta | 8392 |
| S. Poona | BAA-1673 |
| S. Tenneessee | 10722 |
| S. enterica indica Ferlac | 43976 |

TABLE 9
Exclusive strains

| Strain | ATCC # |
|---|---|
| Escherichia coli | 25922 |
| Klebsiella aerogenes | 13048 |
| Klebsiella oxytoca | 13182 |
| Citrobacter freundii | 6879 |
| Proteus hauseri | 13315 |
| Serratia marcescens | 8100 |

Lysis and amplification of the samples were performed as follows, 100 μL of the desired dilutions were added to cluster tubes containing lyophilized pre-treatment mixture. The samples were incubated at 37° C. for 10 minutes. The 100 μL of pre-treatment reaction was transferred to cluster tubes containing a lyophilized chelating resin (Chelex) mix. Then, 400 μL of lysis enzyme solution was added to the same tubes. Samples were vortexed at 2,500 rpm to mix and incubated at 37° C. for 20 minutes. Samples were vortexed again at 2,500 rpm and incubated at 95° C. for 8 minutes. After the incubation, samples were removed from the heat block and 50 μL of the lysed samples were added to PCR tube strips containing lyophilized Salmonella tHDA reaction mixtures. The samples were capped and vortexed to mix and transferred to an Optigene isothermal reader and processed using the Salmonella tHDA assay (pre-incubation: 37° C. for 20 minutes; amplification: 65° C. for 45 minutes with fluorescence measurement every 30 seconds). Components of the pre-treatment mixture, lysis mixture and tHDA reaction mixture are listed in Tables 10-12.

TABLE 10
Pre-treatment mixture components (final concentrations
after resuspension of lyophilized mixture)

| Component | Concentration | |
|---|---|---|
| Tris-HCl pH 8.8 | 10.6 | mM |
| CaCl₂ | 4.1 | mM |
| Micrococcal Nuclease | 0.22 | Units/μL |
| BSA | 0.52 | mg/mL |
| Sucrose | 6.86% | (m/v) |
| Dextran | 1.29% | (m/v) |

TABLE 11
Lysis mixture components (final concentrations for lysis)

| Component | Concentration | |
|---|---|---|
| Tris HCl pH 8.8 | 5.7 | mM |
| Chelex-100 | 4.5% | (m/v) |
| Sucrose | 3.0% | (m/v) |
| Lysozyme | 0.8 | mg/mL |
| Proteinase K | 0.8 | mg/mL |
| Mutanolysin | 20 | Units/mL |
| EGTA | 2.6 | mM |
| Achromopeptidase | 85.6 | Units/mL |
| Dextran | 0.56% | (m/v) |

TABLE 12
tHDA reaction mixture components (final concentrations
after resuspension of lyophilized mixture)

| Component | Concentration | |
|---|---|---|
| Tris-HCl pH 8.8 | 20.4 | mM |
| KCl | 10 | mM |
| NaCl | 24.8 | mM |
| dGTP | 0.5 | mM |
| dCTP | 0.5 | mM |
| dTTP | 0.2 | mM |
| dATP | 6.6 | mM |
| dUTP | 0.3 | mM |
| Sal HDA Forward Primer | 80.7 | nM |
| Sal HDA Reverse Primer | 80.7 | nM |
| Sal HDA ROX Probe | 80.7 | nM |
| Control HDA HEX Probe | 80.7 | nM |
| WarmStart Gst DNA Polymerase | 2 | U/μL |
| Tte-UvrD Helicase | 11.5 | ng/μL |
| Sso-SSB | 2 | ng/μL |
| NxtScript reverse transcriptase | 0.4 | U/μL |
| RNaseH2 | 5.8 | ng/μL |
| Antarctic thermolabile UDG | 0.02 | U/μL |
| Endonuclease VIII | 0.4 | U/μL |
| cPRC (control RNA) | 99,600 | copies/μL |
| DTT | 2 | mM |
| Tween-20 | 0.14% | (v/v) |
| Sucrose | 2% | (m/v) |
| Dextran | 2.5% | (m/v) |
| Magnesium sulfate | 10 | mM |
| BSA | 0.5 | mg/mL |

Table 13 provides the sequences of the control RNA and control HDA HEX probe referred to in Table 12. The term “5HEX” in Table 13 represents 5′ Hexachlorofluorescein, a fluorophore attached to the 5′ end of the oligonucleotide. The term “3BHQ_1” in Table 13 represents 3′ Black Hole Quencher, a quencher attached to the 3′ end of the oligonucleotide that can absorb the fluorescence from the fluorophore (HEX) while the probe is uncleaved. When the probe is cleaved, the quencher is not close enough to absorb fluorescence so the fluorophore's fluorescence can be detected.

TABLE 13
Sequences of control RNA and
control HDA HEX probe

| | | SEQ | |
|---|---|---|---|
| | | ID | |
| Component | Sequence | NO: | Notes |
| Control | AAA AAG GAG AAG | 8 | All bases are RNA. |
| RNA | GCA CGC UGA CAC | | Each base in the |
| | AAA CAG CCA AAU | | sequence is a 2′ |
| | CUA ACC AAC UUU | | fluorinated RNA |
| | ACA CUA CUA GAG | | base (this prevents |
| | AGU GAA GAG AGC | | degradation of the |
| | AGA ACG AUA UUU | | RNA by |
| | ACU CAC GGA GCU | | endonucleases). |
| | GAA GUC AGG ACA | | The first five and |
| | CUA GCC CAA UCA | | last five RNA bases |
| | ACC AAG CAC UAA | | in the sequence are |
| | AAA | | connected by |
| | | | phosphorothioate |
| | | | linkages (which |
| | | | prevents degradation |
| | | | of the RNA by |
| | | | exonucleases). |
| Control | 5′/5HEX/AGA GAG | 9 | Where ‘r’ indicates |
| HDA | TGA AG rA GAG | | an RNA |
| HEX | CAG AAC GA/ | | base. |
| Probe | 3BHQ1/3′ | | |

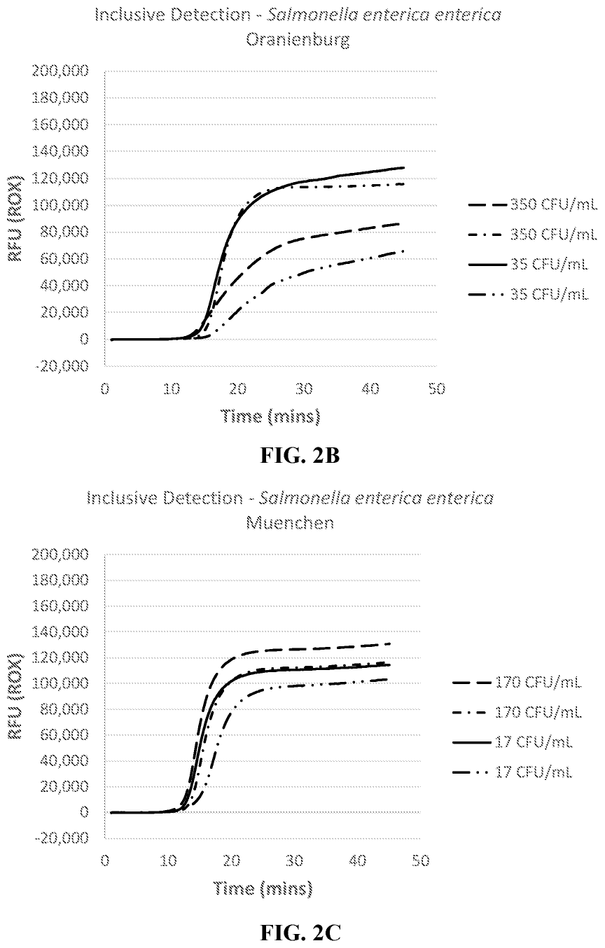

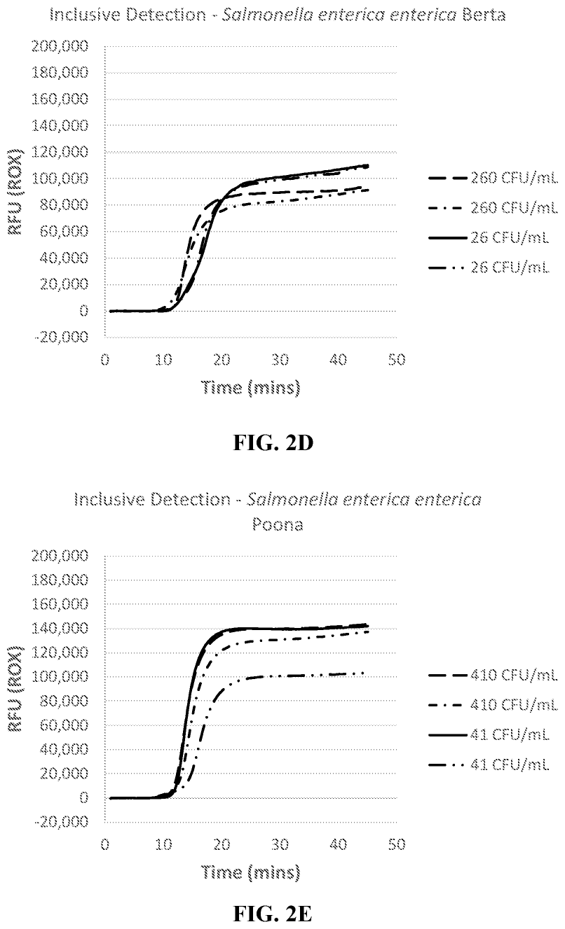

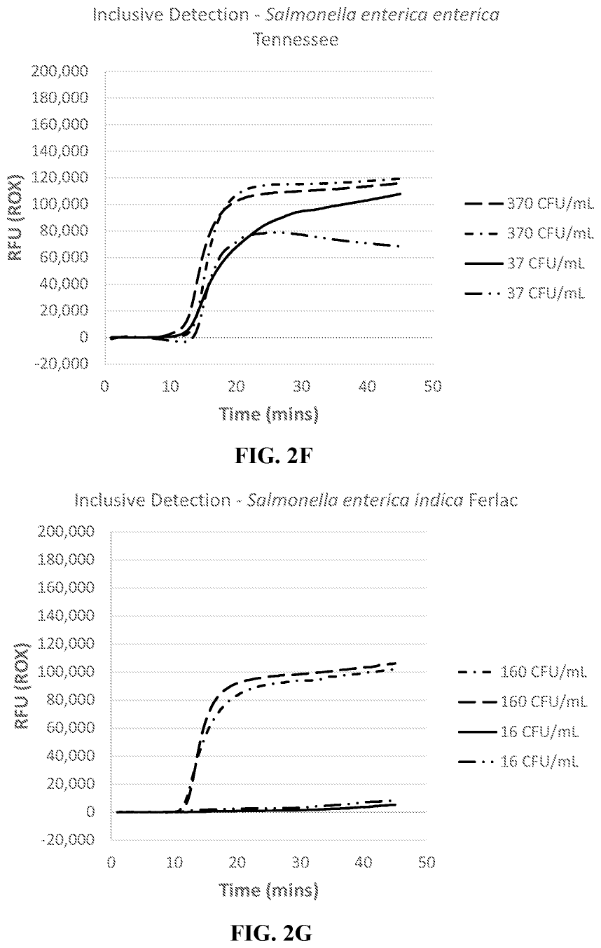

As shown in FIG. 2A-FIG. 2G, amplification signals were observed for the tested inclusive strains. All inclusive strains were detected at 160 CFU/mL or above, with many strains detected between 17 CFU/mL-160 CFU/mL.

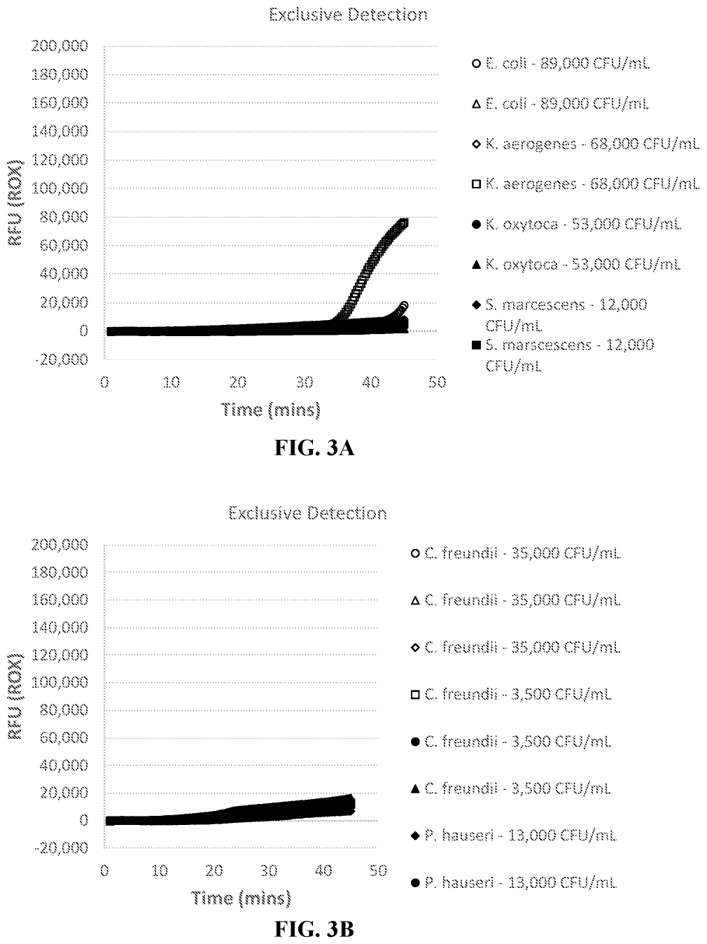

As shown in FIG. 3A-FIG. 3B, tests with the exclusive strains showed no amplification signal or late amplification for some samples that occurred after more than 30 minutes.

As demonstrated by the present example, the designed primer pairs and probe show good specificity and sensitivity for Salmonella detection. The below additional examples illustrate Salmonella detection assays using these primers and probe.

### Example 2. Analysis of Salmonella Detection Assay Sensitivity

Known levels of overnight culture were spiked directly on polymer tip swabs with Letheen Broth (Neogen catalog #6649, also referred to herein as “Letheen swabs”) or polyurethane sponges pre-moistened with 10 mL Hi Cap Neutralizing Buffer (Neogen catalog #36002). For swab collection, the swab was placed back into its original tube after sampling and vortexed to detach target cells into the liquid. After vortexing, 100 μL of sample was transferred to pre-treatment tubes and the Salmonella detection assay procedures were followed. For sponge collection, the following procedure was followed: the sponge was placed back into its original bag after sampling, 15 mL of Butterfield Phosphate Buffer (BPB) was added, the bag was stomached to detach the target cells into the liquid, the samples were concentrated by centrifugation at 5000 rpm for 10 minutes and the supernatant was discarded, the pellet was resuspended with 110 μL BPB, 100 μL of sample was transferred to pre-treatment tubes and the Salmonella detection assay procedures were followed.

For both the swab and the sponge collection methods, the Salmonella detection assay procedures for the present example were as follows: (1) pipette 100 μL of sample into a tube with lyophilized pretreatment mixture (the lyophilized pre-treatment mixture comprises Tris-HCl, CaCl₂, micrococcal nuclease, BSA, sucrose and dextran); (2) vortex 2-3 seconds at half speed or 2,500 rpm; (3) incubate sample for 10 minutes at 37° C. (pre-treatment step); (4) rehydrate lysis components with 14 mL of resuspension buffer (the lysis components and resuspension buffer comprise Tris-HCl, sucrose, lysozyme, proteinase K, mutanolysin, EGTA, achromopeptidase and dextran); (5) add 400 μL of rehydrated lysis buffer to the lyophilized Chelex tube (the Chelex tube comprises Chelex-100, sucrose, dextran, and Tris-HCl); (6) transfer 100 μL of pretreatment sample to the same Chelex tube; (7) vortex 10-15 seconds at half speed or 2,500 rpm; (8) incubate samples for 20 minutes at 37° C. (lysis step); (9) vortex 10-15 seconds at half speed or 2,500 rpm; (10) incubate samples for 8 minutes at 95° C. (lysis inactivation step); (11) take 50 μL of sample from the top of the lysate and transfer it to a lyophilized Salmonella HDA reaction detection mixture (the lyophilized Salmonella HDA reaction detection mixture comprises Tris-HCl, KCl, NaCl, dGTP, dCTP, dTTP, dATP, dUTP, Sal HDA forward primer, Sal HDA reverse primer, Sal ROX Test probe, control HDA HEX probe, WarmStart Gst DNA Polymerase, Tte-UvrD Helicase, Sso-SSB, NxtScript reverse transcriptase, RNaseH2, Antarctic thermolabile UDG, Endonuclease VIII, cPRC (control RNA), DTT, Tween-20, sucrose, dextran, magnesium sulfate, and BSA); (12) vortex 2-3 seconds; (13) spin down 2-3 seconds or tap to settle sample and remove bubbles; and (14) run on Optigene reader.

Table 14 shows results for the swab collection method and Table 15 shows results for the sponge collection method.

TABLE 14
Salmonella detection assay sensitivity with swab collection method

| Organism | Inoculum (CFU) | tHDA (pos/total) |
|---|---|---|
| S. Newport | 23 | 5/8 |
| S. Heidelberg | 29 | 7/8 |
| S. Blockley | 19 | 6/8 |
| S. Heidelberg | 29 | 3/8 |
| S. Newport | 44 | 11/16 |
| S. Newport | 39 | 5/8 |
| S. Blockley | 40 | 7/8 |
| S. Newport | 68 | 4/8 |
| S. Heidelberg | 47 | 4/8 |
| S. Blockley | 50 | 6/8 |
| Overall | 19-68 | 58/88 (66%) |
| S. Heidelberg | 117 | 7/8 |
| S. Typhimurium | 197 | 4/4 |
| S. Blockley | 124 | 8/8 |
| S. Newport | 118 | 8/8 |
| S. Heidelberg | 150 | 8/8 |
| Overall | 117-197 | 35/36 (97%) |

TABLE 15
Salmonella detection assay sensitivity
with sponge collection method

| Organism | Inoculum (CFU) | tHDA (pos/total) |
|---|---|---|
| S. Newport | 113 | 8/8 |
| S. Newport | 56 | 8/8 |
| S. Heidelberg | 61 | 8/8 |
| S. Heidelberg | 146 | 8/8 |
| S. Heidelberg | 146 | 16/16 |
| S. Newport | 98 | 8/8 |
| S. Newport | 98 | 8/8 |
| S. Blockley | 47 | 8/8 |
| S. Blockley | 40 | 8/8 |
| Overall | 40-146 | 80/80 (100%) |

Results for the swab method of collection indicated the assay had a sensitivity of around 66% when the inoculum levels were between 19-68 CFU and 97% when inoculum levels were between 117-197 CFU. Results for the sponge method of collection indicated that the assay had a sensitivity of around 100% for inoculum levels of 40-146 CFU.

### Example 3: Salmonella Detection with Delayed Sample Processing

The present example demonstrates that collected samples do not need to be processed immediately following collection. Instead, collected samples can be temporarily stored prior to the target bacteria detection assay.

Known levels (˜40-80 CFU) of overnight culture was spiked directly on swabs or sponges and held for different times/temperatures prior to following the Salmonella detection assay procedures outlined above in Example 2. Results are shown in Table 16.

TABLE 16
Detection of Salmonella with delayed sample processing

| | Sample | | Inoculum | tHDA |
|---|---|---|---|---|
| Organism | Type | Sample Condition | (CFU) | (pos/total) |
| S. Newport | swab | Room Temperature | 44 | 6/8 |
| | | 3 h | | |
| S. Newport | swab | 4° C. for 3 h | 44 | 5/8 |
| S. Newport | swab | 4° C. for 27 h | 44 | 12/16 |
| S. Typhimurium | sponge | Room Temperature | 74 | 8/8 |
| | | 2 h | | |
| S. Typhimurium | sponge | 4° C. for 2 h | 74 | 8/8 |
| S. Typhimurium | sponge | 4° C. for 26 h | 74 | 8/8 |

As shown by the present example, the Salmonella detection assay can be used to detect target bacteria in samples collected and stored prior to processing. Also as shown here, samples kept cold under refrigerated conditions can be tested within 24 hours.

### Example 4: Salmonella Environmental Surface Testing with Swab Collection

Plastic surfaces (1″×1″) were inoculated with S. Heidelberg and E. coli at 10 times higher as the background microflora. The surfaces were dried overnight at room temperature, and sampled with Letheen swabs. The Letheen swabs were tested by Salmonella detection assay according to the procedures outlined above in Example 2. The reference culture method was also tested. The reference culture method included enrichment in 10 mL lactose broth at 35° C. for 24 hours and enrichment in 10 mL RV broth at 35° C. for 24 hours followed by streaking on a XLD agar plate for identifying typical Salmonella colonies. Results are shown in Table 17.

TABLE 17
Plastic surface (1″ × 1″) with S. Heidelberg and E. coli

| | | | tHDA | Reference |
|---|---|---|---|---|
| | | Inoculum | Result | Result |
| Environmental | | CFU/ | (Positive/ | (Positive/ |
| sample (1 × 1) | Organism | coupon | total) | total) |
| Plastic/swab | S. Heidelberg/ | 34/525 | 8/20 | 13/20 |
| | E. coli | 340/5250 | 5/5 | 5/5 |
| | | 0 | 0/5 | 0/5 |

Although the surface was inoculated with 34 CFU of Salmonella, after overnight drying, most of them were died off, indicated by the fractional positive with reference culture method. Higher number of E. coli was co-inoculated to demonstrate detection of Salmonella in the presence of background microflora. No significant difference was found between tHDA and reference culture method for detecting Salmonella from environmental surfaces.

### Example 5: Salmonella Environmental Surface Testing with Sponge Collection

Stainless steel surfaces (4″×4″) were inoculated with S. Heidelberg. The surfaces were dried overnight at room temperature, and sampled with pre-moistened sponges. The sponge samples were tested by Salmonella detection assay according to the procedures outlined above in Example 2. The reference culture method was also tested. The reference culture method included enrichment in 225 mL lactose broth at 35° C. for 24 hours followed by streaking on a XLD agar plate for identifying typical Salmonella colonies. Results are shown in Table 18.

TABLE 18
Stainless steel surface (4″ × 4″) with S. Heidelberg

| | | | tHDA | Reference |
|---|---|---|---|---|
| | | Inoculum | Result | Result |
| Environmental | | CFU/ | (Positive/ | (Positive/ |
| sample (4 × 4) | Organism | coupon | total) | total) |
| Stainless Steel/ | S. Heidelberg | 140 | 2/10 | 2/10 |
| sponge | | 420 | 5/10 | 4/10 |

As demonstrated herein with both swab and sponge samples, the tHDA assay with pre-treatment is comparable to the conventional culture method in detecting Salmonella spp. on environmental surfaces.

### Example 6: Salmonella Detection from Environmental Surface Samples without Pre-Treatment Incubation

If the pre-treatment incubation at 37° C. for 10 minutes is skipped, tHDA shows more positive results, as shown in Table 19. The purpose of pre-treatment is to remove nucleic acids from lysed cells existing in the environment. Without pre-treatment, most free nucleic acids are not cleaved, therefore more positive results are observed.

TABLE 19
Environmental surface without pre-treatment incubation

| | | | | tHDA |
|---|---|---|---|---|
| | | Inoculum | | Result |
| Environmental | | CFU/ | | (Positive/ |
| sample (1 × 1) | Organism | coupon | Pre-treatment | total) |
| Plastic/swab | S. Blockley | 149 | 37° C. 10 min | 2/8 |
| | | | No incubation | 8/8 |

### Example 7: Alternative Sample Preparation Protocol and Detection of Salmonella Target Nucleic Acids

The present example demonstrates an alternative method for preparing samples to detect nucleic acids from Salmonella species (spp.) without using pre-treatment, lysis enzymes, or chelating resin.

Inclusivity and No Template Control (NTC) Testing

An overnight culture of Salmonella Typhimurium was prepared by growing it in Tryptic Soy Broth (TSB) at 37° C. The culture was serially diluted in Molecular-grade water to obtain the desired dilution. Immediately, 100 μL of the desired dilutions were plated on Tryptic Soy Agar (TSA) plates and incubated overnight at 37° C. to obtain the titers for the overnight culture.

Lysis and amplification of the samples were performed as follows, 100 μL of the desired dilutions were added to 1.5 mL micro-centrifuge tubes. 400 μL of Molecular-grade water was added to each dilution. No template controls (NTCs) substituted 100 μL of Molecular-grade water in the place of diluted culture. The samples were incubated at 80° C. for 10 minutes. After the incubation, samples were removed from the heat block and 50 μL of the lysed samples were added to PCR tube strips containing lyophilized Salmonella tHDA reaction mixtures. The samples were capped and vortexed to mix and transferred to Bio-Rad CFX96 qPCR instrument and processed using an isothermal program (pre-incubation: 37° C. for 20 minutes; amplification: 65° C. for 30.5 minutes with fluorescence measurement every 30 seconds). Components of the tHDA reaction mixture are listed in Table 20.

TABLE 20
tHDA reaction mixture (final concentrations
after resuspension of lyophilized mixture)

| Component | Concentration | |
|---|---|---|
| Tris-HCl pH 8.8 | 20.0 | mM |
| KCl | 10.0 | mM |
| NaCl | 40.0 | mM |
| dGTP | 0.5 | mM |
| dCTP | 0.5 | mM |
| dTTP | 0.2 | mM |
| dATP | 6.5 | mM |
| dUTP | 0.3 | mM |
| Sal HDA Forward Primer | 80 | nM |
| Sal HDA Reverse Primer | 80 | nM |
| Sal ROX Test Probe (5′/56-ROXN/CCA TGA GTA | 80 | nM |
| AAT rCAC TTC ACC TAC GTG/3IAbRQSp/3′) | | |
| Control HDA HEX Probe | 80 | nM |
| WarmStart Gst DNA Polymerase | 2 | U/μL |
| Tte-UvrD Helicase | 10.0 | ng/μL |
| Sso-SSB | 2 | ng/μL |
| NxtScript reverse transcriptase | 0.4 | U/μL |
| RNaseH2 | 5.0 | ng/μL |
| Antarctic thermolabile UDG | 0.02 | U/μL |
| cPRC (control RNA) | 700 | copies/μL |
| DTT | 2 | mM |
| Tween-20 | 0.14% | (v/v) |
| Sucrose | 2% | (m/v) |
| Dextran | 2.5% | (m/v) |
| Magnesium sulfate | 10 | mM |
| BSA | 0.5 | mg/mL |

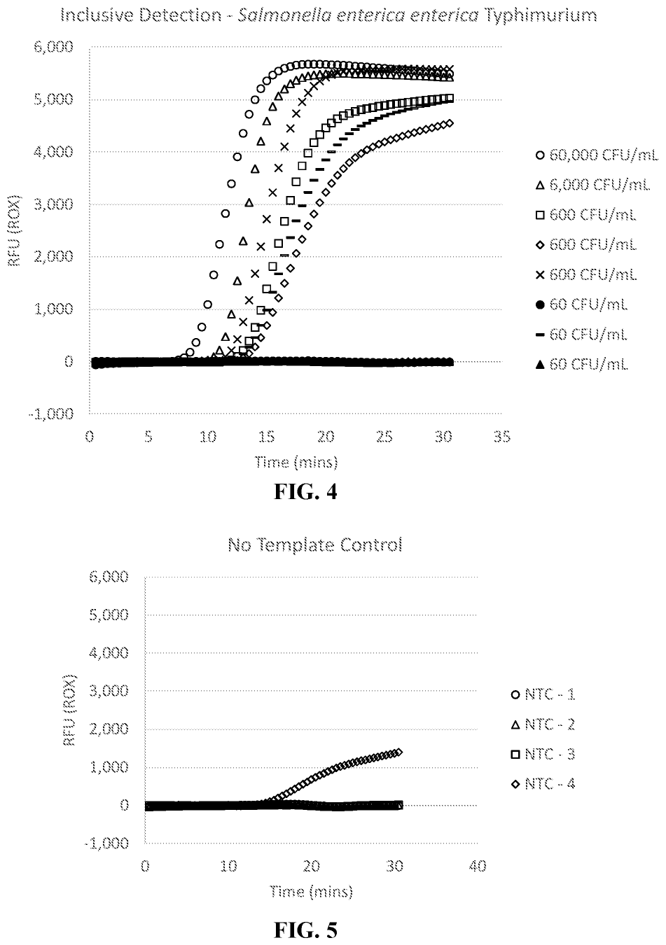

As shown in FIG. 4, amplification signals were observed for S. Typhimurium. All samples at 600 CFU/mL or above were detected, with partial detection (⅓) at 60 CFU/mL. These results indicate that the assay limit of detection using this method is between 60-600 CFU/mL and that HDA mechanism can detect Salmonella spp. without the use of pre-treatment and a simple heating lysis protocol.

As shown in FIG. 5, tests with no Salmonella cells present (NTC) showed no amplification signal or weak and late amplification.

As demonstrated by the present example, the designed primer pairs and probe show good specificity and sensitivity for Salmonella detection.

What is claimed is:

1-73. (canceled)

74. A method for determining the presence or absence of target bacteria in a sample, the method comprising the steps of:
(i) providing a sample;
(ii) contacting an aliquot of the sample with a lysis mixture under conditions to lyse at least a portion of cells in the aliquot, thereby generating a lysate;
(iii) contacting an aliquot of the lysate with a detection mixture, thereby generating an assay mixture;
(iv) in the assay mixture, reverse transcribing a target RNA of the target bacteria to form target cDNA and amplifying the target cDNA by helicase-dependent amplification (HDA); and
(v) detecting presence or absence of the amplified target cDNA, thereby determining the presence or absence of the target bacteria in the sample.

75. The method of claim 74, wherein the method does not include enrichment for cells in the sample.

76. The method of claim 74, wherein the method does not include a prolonged incubation period to increase concentration of the target RNA prior to contacting the sample with the lysis mixture.

77. The method of claim 74, wherein the sample is an environmental sample.

78. The method of claim 74, wherein the sample is collected from an environment comprising a low concentration of the target bacteria cells.

79. The method of claim 74, wherein the method is of sufficient sensitivity to detect the presence of the target bacteria from a sample having as little as about 30-60 colony forming units (CFU) of the target bacteria.

80. The method of claim 74, wherein the target RNA comprises a Salmonella RNA sequence of the 23S ribosomal RNA.

81. The method of claim 74, further comprising
collecting the sample to be provided in step (i), wherein the sample is collected from an environment that is being tested for bacterial contamination by the target bacteria.

82. The method of claim 74, further comprising
pre-treating an aliquot of the sample to remove nucleic acids not associated with intact cells, thereby generating a pre-treated sample; and
wherein the step of contacting the aliquot of the sample with the lysis mixture comprises contacting an aliquot of the pre-treated sample with the lysis mixture under conditions to lyse at least a portion of cells in the aliquot of the pre-treated sample.

83. The method of claim 82, wherein:
the step of pre-treating the aliquot of the sample comprises contacting the aliquot of the sample with a pre-treatment mixture under conditions to remove nucleic acids not associated with intact cells in the aliquot of the sample, and wherein the pre-treatment mixture comprises a nuclease that cleaves nucleic acids and the lysis mixture comprises a component that inactivates the nuclease.

84. The method of claim 74, wherein the step of detecting comprises measuring a fluorescent readout indicative of the presence of the amplified target cDNA.

85. The method of claim 74, wherein the detection mixture comprises at least one probe for detecting the amplified target cDNA.

86. The method of claim 85, wherein the detection mixture further comprises a helicase, an energy source for the helicase, a DNA polymerase, a reverse transcriptase, and dNTPs.

87. The method of claim 86, wherein the detection mixture further comprises (i) an enzyme that binds uracil in a DNA strand and converts it into an apurinic site; (ii) an enzyme that cleaves DNA at apurinic sites; and (iii) a specialized dNTP that is recognized by the enzyme of (i).

88. The method of claim 74, wherein the provided environmental sample has been stored at 4° C. for up to 24 hours.

89. A kit comprising a first mixture and a second mixture, wherein said first mixture comprises micrococcal nuclease and a divalent salt and said second mixture comprises a divalent ion chelator, at least one lytic enzyme and at least one protease.

90. A kit comprising a first primer having hybridization specificity for a single-stranded nucleic acid region comprising a nucleic acid sequence of Salmonella 23S ribosomal RNA and a second primer having hybridization specificity for a single-stranded nucleic acid region comprising a nucleic acid sequence complementary to the nucleic acid sequence of the Salmonella 23S ribosomal RNA.

91. The kit of claim 90, further comprising at least one probe for detecting a nucleic acid molecule comprising the nucleic acid sequence of:
(i) CAC GTA GGT GAA GTG ATT TAC TCA CGG (SEQ ID NO: 6), or a sequence with at least 90% sequence identity to SEQ ID NO: 6; or
(ii) CAC GTA GGT GAA GTG ATT TAC TCA TGG (SEQ ID NO: 7), or a sequence with at least 90% sequence identity to SEQ ID NO: 7.

92. A primer comprising or consisting of the nucleic acid sequence of GAG AAG GCA CGC TGA CAC (SEQ ID NO: 2) or a sequence with at least 90% sequence identity to SEQ ID NO: 2.

93. A primer comprising or consisting of the nucleic acid sequence of CTG ACT TCA GCT CCG TGA GTA AAT (SEQ ID NO: 3) or a sequence with at least 90% sequence identity to SEQ ID NO: 3.

94. A lyophilized composition comprising micrococcal nuclease and calcium chloride (CaCl₂), wherein, upon resuspension of the lyophilized composition, the concentration of micrococcal nuclease ranges from 0.1-0.3 Units/μL and the concentration of CaCl₂ ranges from 2-6 mM.

95. A lyophilized composition comprising (i) at least one of lyosozyme and mutanolysin;
(ii) at least one of proteinase K and achromopeptidase; and (iii) EGTA, wherein, upon resuspension of the lyophilized composition, the concentration of lysozyme ranges from 0-1 mg/mL, the concentration of mutanolysin ranges from 0-30 Units/mL, the concentration of proteinase K ranges from 0-1 mg/mL, the concentration of achromopeptidase ranges from 0-150 Units/mL, and the concentration of EGTA ranges from 2-5 mM.


SEQUENCE LISTING (9 sequences)

SEQ ID NO: 1 - RNA, 54 nt, Salmonella typhimurium
gagaaggcacgcugacacguaggugaagugauuuacucacggagcugaagucag

SEQ ID NO: 2 - DNA, 18 bp, Artificial Sequence
  primer
gagaaggcacgctgacac

SEQ ID NO: 3 - DNA, 24 bp, Artificial Sequence
  primer
ctgacttcagctccgtgagtaaat

SEQ ID NO: 4 - DNA, 54 bp, Artificial Sequence
  amplicon
gagaaggcacgctgacacgtaggtgaagtgatttactcacggagctgaagtcag

SEQ ID NO: 5 - DNA, 27 bp, Artificial Sequence
  probe
  misc_RNA (13)..(13): RNA base
ccatgagtaaatcacttcacctacgtg

SEQ ID NO: 6 - DNA, 27 bp, Artificial Sequence
  probe hybridization region of amplicon
cacgtaggtgaagtgatttactcacgg

SEQ ID NO: 7 - DNA, 27 bp, Artificial Sequence
  probe hybridization region of amplicon
cacgtaggtgaagtgatttactcatgg

SEQ ID NO: 8 - RNA, 135 nt, Artificial Sequence
  control RNA
aaaaaggagaaggcacgcugacacaaacagccaaaucuaaccaacuuuacacuacuagagagugaagagagcagaacgauauuuacucacggagcugaagucaggacacuagcccaaucaaccaagcacuaaaaa

SEQ ID NO: 9 - DNA, 23 bp, Artificial Sequence
  probe
  misc_RNA (12)..(12): RNA base
agagagtgaagagagcagaacga